(19) United States
(12) Patent Application Publication
(10) Pub. No.: US 2025/0101451 A1
(43) Pub. Date: Mar. 27, 2025

(54) METHOD FOR PREPARING BROWN PLANTHOPPER RESISTANT PLANTS USING RICE BROWN PLANTHOPPER RESISTANCE GENE BPH33.2

(71) Applicant: Fujian Agriculture And Forestry University, FUZHOU (CN)

(72) Inventors: JIANGUO WU, FUZHOU (CN); JIE HU, FUZHOU (CN); SHUAI ZHANG, FUZHOU (CN); SHANSHAN ZHAO, FUZHOU (CN); XIAOQING WU, FUZHOU (CN)

(21) Appl. No.: 18/414,498

(22) Filed: Jan. 17, 2024

(30) Foreign Application Priority Data
Jul. 21, 2023 (CN) 202310900814X

(51) Int. Cl. C12N 15/82; A01H 1/04; C12Q 1/6895

(52) U.S. Cl. CPC C12N 15/8286; A01H 1/045; C12N 15/8205; C12Q 1/6895; C12Q 2600/13

(57) ABSTRACT

The present disclosure locates a new brown planthopper resistance gene BPH33.2 on the short arm of rice chromosome 4 by extreme mixed pool analysis (BSA-seq), and genetically transforming the gene BPH33.2 to make the susceptible rice to be the brown planthopper resistant phenotype rice; at the same time, the knockout of the gene BPH33.2 was used to cause the loss of the brown planthopper resistant phenotype in insect resistant rice, thus confirming the function of BPH33.2. The present disclosure also provides molecular markers closely linked with the rice brown planthopper resistance gene BPH33.2. Through experimental detection of molecular markers linked or co-segregated with these resistance loci, the brown planthopper resistance of rice plants can be accurately predicted at the seedling stage, accelerating the progress of the selection of brown planthopper-resistant rice varieties.

### CROSS-REFERENCE TO RELATED APPLICATIONS

This application is based upon and claims priority to Chinese Patent Application No. 202310900814.X, filed on Jul. 21, 2023, the entire contents of which are incorporated herein by reference.

This application contains a Sequence Listing in form of XML electronically filed and hereby incorporated by reference into the specification in its entirety. The name of the XML file containing the Sequence Listing is brown planthopper resistance gene BPH33.2.xml. The size of the XML file is 29,886 bytes, and the XML file was created on Sep. 14, 2023.

### TECHNICAL FIELD

The present disclosure relates to the technical field of plant genetic engineering, in particular to a rice brown planthopper resistance gene BPH33.2 and its closely linked molecular markers, and the application of the molecular markers in breeding and variety improvement for brown planthopper resistance in rice.

### BACKGROUND

Rice (Oryza sativa) is one of the most important food crops in the world, and the stability of its yield is crucial for securing the national economy and people's livelihood. As a model research crop in the grass family, rice has a relatively small genome, very mature transgenic technology, and increasingly in-depth functional genome research. Therefore, rice has shown unrivalled superiority and importance in socio-economic development, national security and scientific research.

Brown planthopper (BPH, Nilaparvata lugens) is the most important pest in rice growing areas. The brown planthopper is highly explosive, migratory, and has a large and deep damage area. According to the China Agricultural Yearbook, Nilaparvata lugens occurs on more than 20 million hectares annually in China, resulting in a loss of about 3 million tonnes of rice every year. The brown planthopper has become a serious threat to the safe production of rice in China.

At present, chemicals are mainly used in production to control brown planthopper. However, the misuse of pesticides not only increases farmers' production and labour costs, but also causes irreversible harm to the environment. At the same time, pesticides can also kill the natural enemies of brown planthopper and enhance the resistance of the pest, which in turn induces the brown planthopper to become rampant again. Breeding insect-resistant varieties using rice resistance genes is currently the most cost-effective prevention and control strategy. The International Rice Research Institute (IRRI) has proved in the practice of brown planthopper prevention and control that planting rice varieties with different resistance can effectively control the crazy growth of brown planthopper population and slow down the mutation rate of its biotypes or pest-causing properties to a certain extent, so as to achieve the purpose of lasting resistance. Therefore, the continuous discovery and cloning of new resistance genes and their application to breeding is a key research topic to address the integrated control of brown planthopper. In addition, the cloning of resistance genes and the elucidation of resistance mechanisms can enrich the results of rice functional genome research.

There are abundant brown planthopper-resistant rice germplasm resources in nature, and the identification and cloning of resistance genes in these resistant rice have been the focus of scientists' research. As of 2022, researchers have identified at least 40 brown planthopper resistance genes or QTLs from both wild and indica rice resources. As rice functional genome research continues, some brown planthopper resistance genes have also been cloned. So far, 15 resistance genes have been cloned in rice. Among them, Chinese research teams have made major contributions, cloned 13 of these genes. Professor He Guangcun's team at Wuhan University cloned the first brown planthopper resistance gene in rice, Bph14, which encodes the motif of a helix-helix-nucleotide binding site leucine repeat (CC-NB-LRR), and whose unique LRR region may exercise an important function in signalling recognition response and activation of the defence response after brown planthopper invasion (Du et al. 2009 PNAS 106:22163-22168).

### SUMMARY

The present disclosure provides a rice brown planthopper resistance gene, BPH33.2, and its molecular markers and applications.

The present disclosure employs forward genetics method to locate the resistance gene BPH33.2 by constructing a genetic population of resistant and susceptible material, the function of BPH33.2 is confirmed by genetically transforming the gene BPH33.2 to give rise to a brown planthopper resistance phenotype in susceptible rice, and by using knockout of the gene BPH33.2 to cause loss of the brown planthopper resistance phenotype in the insect-resistant rice.

The present disclosure provides a rice brown planthopper resistance gene BPH33.2, the nucleotide sequence of said gene being as shown in SEQ ID NO.1. The gene has a total length of 8,674 bp with four exons and three introns, a first exonic region of 2514-2778 bp, a second exonic region of 3456-3724 bp, a third exonic region of 4535-7732 bp, and a fourth exonic region is 7940-8674 bp. Further, the cDNA of said rice brown planthopper resistance gene BPH33.2 has a full length of 4470 bp, with a sequence as shown in SEQ ID NO.2, encoding 1072 amino acids. The present disclosure also provides a protein encoded by said rice brown planthopper resistance gene BPH33.2, the amino acid sequence of said protein being as shown in SEQ ID NO.3.

The present disclosure also provides molecular markers tightly linked to the rice brown planthopper resistance gene BPH33.2, obtained by conventional PCR amplification with primer pairs of one of the following:

### 1) H99 Marker Primers:

left end primer sequence:
5′-CACTGTGGTTACAACAGAGGT-3′.
right end primer sequence:
5′-TCTCTTCTCGTTGCTGCTCA-3′;

### 2) H79 Marker Primers:

left end primer sequence:
5′-CCGTGAGTTCACTTGTAA-3′,
right end primer sequence:
5′-GTACGATTTGACCAGCGAG-3′;

### 3) 33-3 Marker Primers:

left end primer sequence:
5′-CCTCGCTTACGAAATTAGTG-3′,
right end primer sequence,
5′-TGTGGCAGAGCAAGAGGG-3′;

### 4) 33-4 Marker Primers,

left end primer sequence,
5′-GCTTTCGGATTATCTTCACTA-3′,
right end primer sequence,
5′-AGCTCCCTAAGCTCAACCA-3′.

The present disclosure also provides a molecular marking method for detecting whether the brown planthopper resistance gene BPH33.2 exist in rice, comprising:

amplifying genomic DNAs of rices to be examined by the marker primers according to claim 10, and detecting amplification products; if a DNA fragment of 96 bp can be amplified by H99 marker primers, or a DNA fragment of 140 bp can be amplified by H79 marker primers, or a DNA fragment of 330 bp can be amplified by 33-3 marker primers, or a DNA fragment of 868 bp can be amplified by 33-4 marker primers, all of them indicate a presence of a brown planthopper resistance locus BPH33.2 in rice insect-resistant varieties; if only a 112 bp DNA fragment can be amplified with H99 marker primers, or only a 148 bp DNA fragment can be amplified with H79 marker primers, or no DNA fragment can be amplified with 33-3 marker primers, or no DNA fragment can be amplified with 33-4 marker primers, all of them indicate an absence of a brown planthopper resistance locus BPH33.2 in rice insect-resistant varieties.

The present disclosure provides a method of breeding plants with brown planthopper resistance using transgenic technology, comprising:
1) transforming polynucleotides containing the gene BPH33.2 into cells of plant callus; said polynucleotide sequence being as shown in SEQ ID NO. 1 or SEQ ID NO. 2;
2) regenerating the transformed plant cells into plants;
3) culturing the regenerated plants and allowing expression of said polynucleotides, and harvesting T1 generation seeds;
4) sowing T1 generation seeds, detecting the gene BPH33.2 using molecular markers 33-3 and 33-4 as described above, and harvesting genetically pure T2 generation seeds.

The present disclosure also provides a method of generating plants with brown planthopper resistance using conventional breeding (non-transgenic), said method comprising crossing a plant having the brown planthopper resistance gene BPH33.2 with other plant to generate a progeny plant with brown planthopper resistance. Wherein said plant is monocotyledonous plant.

### BRIEF DESCRIPTION OF THE DRAWINGS

FIG. 1 is the brown planthopper resistance phenotype of the BPH33 near isogenic line. A,B, seedling resistance in near isogenic lines; C,D, area of honeydew secreted by brown planthopper on near isogenic lines stained with ninhydrin (purple-red part). 9311 and NIL-BPH33 respectively denote near isogenic lines that do not carry and carry the gene BPH33 in purity; scale bar: 5 cm.

FIG. 2 is the fine positioning of BPH33. The arrow above indicates the molecular marker, the number below is the physical position of the corresponding molecular marker on the chromosome, and numbers in parentheses represent the number of individual plants whose genotypes and phenotypes are inconsistent with each other on the corresponding molecular markers; white boxed columns represent pure 9311 genotypes, black columns represent pure KOLAYAL (KOL) genotypes, and grey columns indicate recombination breakpoints; n is the number of individual plants contained in the segregating population used for screening the recombinant individual plants, and 16-100 are the number of recombinant individual plants, and the right side represents the phenotypes of the recombinant individual plants for seedling brown planthopper resistance, S and R represent respectively susceptible and resistant, and smaller resistance scores denoting greater resistance.

FIG. 3 is a schematic diagram of the BPH33 candidate gene region.

FIG. 4 is the phenotype of the knockout lines for the BPH33 candidate gene. A, seedling resistance of the BPH33.1/BPH33.3 double knockout mutant; B, seedling resistance of the BPH33.2/BPH33.3 double knockout mutant; C, seedling resistance of the BPH33.2 single knockout mutant.

FIG. 5 is the phenotype of the BPH33 candidate gene background expression transgenic family line T2. A, the seedling resistance of family line T2 with BPH33.1 background expression transgene; B, the seedling resistance of family line T2 with BPH33.3 background expression transgene; C, seedling resistance of family line T2 with BPH33.2 background expression transgene.

FIG. 6 is a gel map of the BPH33.2 molecular marker. M, DNA gradient marker; lanes 1-8 are eight samples from the F2 population, and 9 and 10 are the resistant donor KOLAYAL and the susceptible control 9311 respectively. A, B, C, and D are gel plots of the markers H99, H79, 33-3, and 33-4, respectively. S, susceptible; R, resistant.

FIG. 7 is the brown planthopper resistance in improved lines of BPH33.2 and their hybrids. A shows brown planthopper seedling resistance in conventional improved lines carrying the pure combination of BPH33.2; and B shows brown planthopper seedling resistance in improved lines of hybrid rice carrying the heterozygous BPH33.2. HZ (BPH3), WSSM (BPH3), G8B (BPH33) and SJX (BPH33) are genetically improved families that introduced BPH33, a brown planthopper resistance gene, into the background of excellent rice varieties huazhan, wushansimiao, guang8B and 19xiang, respectively. HZ and SJX are control species huazhan and wushansimiao, respectively, that do not contain the brown planthopper resistance gene. 9311/Y58S (BPH33) is the hybrid combinations of rice restorer line 9311 carrying homozygous gene BPH33 and two-line male sterile lines Y58S. 9311/GZ63SS (BPH33) is the hybrid combinations of rice restorer line 9311 carrying homozygous gene BPH33 and Guangzhan 63S. 9311/GZ63SS is the hybrid combination without BPH33 and is the control.

FIG. 8 is a flow chart of the transgenic method of preparing plant with brown planthopper resistance.

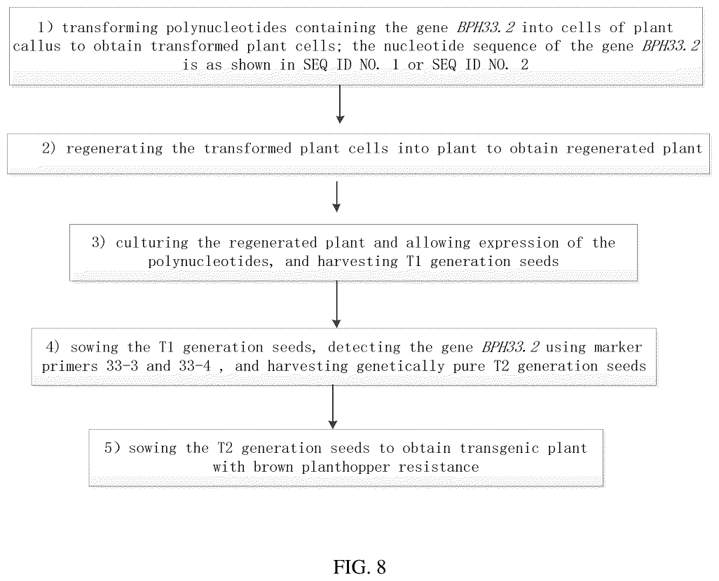

### DETAILED DESCRIPTION OF THE EMBODIMENTS

In order to further clarify the purpose, technical solution and advantages of the present disclosure, the present disclosure will be specified below in conjunction with the accompanying drawings and specific implementations. Some specific details are discussed in the following description to facilitate full understanding of the present disclosure. However, the present disclosure may also be implemented in many other ways different from those described herein, and those skilled in the art may make similar improvements without departing from the spirit of the present disclosure. Thus, the present disclosure is not intended to be limited by the specific implementations disclosed below.

Unless otherwise defined, all technical and scientific terms used herein have the same meaning as commonly understood by one of ordinary skill in the technical field of this disclosure. The terminologies used in the description of the present disclosure are merely for the purpose of describing the specific embodiments, and are not intended to limit the scope of the present disclosure.

Various definitions within the scope of the disclosure used in this specification are explained in detail below.

A BPH-susceptible Chinese indica rice cultivar 9311 was crossed with two BPH-resistant Sri Lankan indica rice cultivars, KOLAYAL (IRGC 36295) and POLIYAL (IRGC 36352), which were kindly provided by the Inter-national Rice Research Institute (IRRI).

For the information of rice varieties R498, Zhenshan 97 (ZS97), Minghui 63 (MH63), Nipponbare (NPB), Huanghuazhan, Huazhan, Wushansimiao, 19xiang, Guang 8B, Guangzhan 63s, Y58S, Zhonghua 11, Kasalath, please refer to the website https://www.ricedata.cn/variety/default_advanced.aspx.

Extreme mixed pool analysis, also known as bulk subgroup analysis (BSA-Seq), it refers to the selection of parents with significant phenotypic differences for the studied phenotypic traits to build a phenotypic segregating population, the selection of a certain number of individuals with extreme phenotypes in the segregating population, the mixing of DNA pools to build DNA pools, and the comparison of gene mutation frequencies between different phenotypic DNA pools. The regions with differences are the chromosomal candidate positions where the target genes or QTLs are located, and the functional annotation of genes in the locked candidate interval, to achieve rapid mapping of trait determining genes. BSA combined with high-throughput genome resequencing technology can efficiently, rapidly and accurately identify trait determining genes, and can significantly reduce research costs. This technology has been widely used in a variety of animal and plant breeding research.

Marker-assisted selection technology (MAS) uses the characteristics of close linkage between molecular markers and genes that determine target traits. By detecting molecular markers, the existence of target genes can be detected, and the purpose of selecting target traits can be achieved. It has the advantages of fast, accurate and free from environmental conditions. It can be used as an auxiliary means to identify parental relationships, transfer of quantitative and recessive traits in backcross breeding, selection of hybrid offspring, prediction of Heterosis and purity identification of varieties. Molecular marker assisted selection (MAS) refers to the selection of target trait genotypes by analyzing the genotypes of molecular markers closely linked to the target gene. Molecular marker assisted breeding shortens the breeding period, accelerates the breeding process, improves the breeding efficiency, and overcomes many difficulties in conventional breeding methods. With the development of some new molecular markers, including disease resistance, insect resistance, drought resistance, high yield, quality improvement and other aspects.

The third-generation sequencing technology refers to single-molecule sequencing technology. When sequencing DNA, it does not need to undergo PCR amplification, realizing the separate sequencing of each DNA molecule. The third-generation sequencing technology is also called de novo sequencing technology, that is, single-molecule real-time DNA sequencing. The inventor of the third-generation sequencing technology is Dr. Stephen w turner1 & Jonas korlach1. Gene sequencing technology has gradually become an important technical means in clinical molecular diagnosis. Three generation sequencing technology is the main development direction in the future. The third generation sequencing technology is applied in genome sequencing, methylation research and mutation identification (SNP detection).

CRISPR/CAS (clustered regularly interspaced short palindromic repeats/CAS) system is a widely used gene editing system. Its principle is that gRNA generated by CRISPR transcription mediates CAS nuclease to target the target sequence and cleave the sequence. Among them, the most commonly used is the DNA targeting crispr/cas9 system, which is modified from the class II crispr/cas system. It can effectively target edit in a variety of cells such as plants, bacteria, yeast, fish and mammals. It has the advantages of simple operation, high efficiency, and many target sites. In crispr/cas9 system, sgRNA (short guide RNA) recognizes and binds the target sequence of target gene, guides cas9 to cut the binding site, and generates DNA double strand break (DSB). The organism itself repairs DSB by non homologous end joining (NHEJ). Proteins involved in repair often insert or delete several bases at the DNA end, The function of the repaired gene is lost due to mutation, thus realizing gene knockout in the body.

FGENESH is one of hundreds of free genetic analysis tools developed by softberry (website link: http://www.softberry.com/berry.pht).

Promoter is a sequence that binds to RNA polymerase and can initiate mRNA synthesis.

Introns are interspersed sequences in eukaryotic cell DNA. These sequences are transcribed in the precursor RNA, removed by splicing, and finally do not exist in the mature RNA molecule. The alternate arrangement of introns and exons constitutes a fragmented gene. Introns in precursor RNA are often referred to as “intervening sequences”. It has more mutations than exons in post transcriptional processing. Intron is a special DNA sequence.

Exons is a part of eukaryotic genes, which will be preserved after splicing and can be expressed as a protein during protein biosynthesis. Exons are the last gene sequences that appear in mature RNA, also known as expression sequences. Nucleotide sequences that exist both in the initial transcripts and in mature RNA molecules. Coding regions are identified by identifying segments that occur in a variety of organisms, and the conservation of exons can serve as the basis for such identification.

CDS (coding sequences) is a DNA sequence corresponding to the protein sequence one by one, and there is no other sequence unrelated to the protein in the middle of the sequence, which is the closest to the real situation.

UTR (Untranslated Regions) are non coding segments at both ends of messenger RNA (mRNA) molecules. The 5′-utr extends from the methylated guanine nucleotide cap at the beginning of the mRNA to the AUG start codon, and the 3′-utr extends from the stop codon at the end of the coding region to the front of the poly-A tail (poly-A).

Previously, our research lab introduced more than 300 varieties or lines from IRRI that may be resistant to a wide range of BPH biotypes. By evaluating seedling resistance, we screened more than 20 highly resistant varieties. Among them, two local indica rice varieties KOLAYAL and POLIYAL from Sri Lanka showed high resistance to brown planthopper in both Fuzhou and Wuhan, China.

In order to locate the resistance gene, we constructed the BC₁F_{2:3} population of insect susceptible varieties 9311 and KOLAYAL, 9311 and poliyal, and simultaneously mapped a new resistance gene BPH33.2 on the short arm (4S) of rice chromosome 4 by using extreme mixed pool sequencing (BSA-Seq). And it was fine mapped to a 30 KB region by recombinant single plant analysis. Through candidate gene analysis and genetic transformation validation, BPH33.2 was finally cloned. Through the map based cloning method of forward genetics, the invention successfully cloned a new resistance gene of Nilaparvata lugens, and developed a series of molecular markers closely linked or co isolated with the resistance gene. With the help of molecular marker assisted selection (MAS) technology, the insect resistance gene can be accurately introduced or aggregated, so as to select durable resistant varieties and effectively inhibit the population of Nilaparvata lugens, Save labor and pesticide costs and increase rice yield.

The present disclosure provides a rice brown planthopper resistance gene, BPH33.2, and its molecular markers and applications, can accurately predict brown planthopper resistance in rice plants at the seedling stage and accelerate the selection of brown planthopper-resistant rice varieties by experimentally detecting molecular markers linked or co-segregated with these resistance loci.

The present disclosure employs forward genetics method to locate the resistance gene BPH33.2 by constructing a genetic population of resistant and susceptible material, the function of BPH33.2 is confirmed by genetically transforming the gene BPH33.2 to give rise to a brown planthopper resistance phenotype in susceptible rice, and by using knockout of the gene BPH33.2 to cause loss of the brown planthopper resistance phenotype in the insect-resistant rice.

The present disclosure provides a rice brown planthopper resistance gene BPH33.2, the nucleotide sequence of said gene being as shown in SEQ ID NO.1. The gene has a total length of 8,674 bp with four exons and three introns, a first exonic region of 2514-2778 bp, a second exonic region of 3456-3724 bp, a third exonic region of 4535-7732 bp, and a fourth exonic region is 7940-8674 bp. Further, the cDNA of said rice brown planthopper resistance gene BPH33.2 has a full length of 4470 bp, with a sequence as shown in SEQ ID NO.2, encoding 1072 amino acids. The present disclosure also provides a protein encoded by said rice brown planthopper resistance gene BPH33.2, the amino acid sequence of said protein being as shown in SEQ ID NO.3.

Because of the concatenation of codons in the coding region of the gene, the present disclosure also comprises substitutions, additions and/or deletions of one or more nucleotides to the polynucleotide sequence encoding said protein having the same function as described above.

It should be understood that without affecting the activity of the BPH33.2 protein, the person skilled in the art may make various substitutions, additions and/or deletions of one or more amino acids to the amino acid sequence shown in SEQ ID NO.3 to obtain amino acid sequences having the same function, which can be applied to the genetic improvement of resistance to rice brown planthopper.

The present disclosure also provides molecular markers tightly linked to the rice brown planthopper resistance gene BPH33.2, the molecular markers are H99, H79, 33-3, 33-4, which are obtained by conventional PCR amplification with primer pairs of one of the following:

### (1) H99 Marker Primers:

left end primer sequence:
(SEQ ID NO. 4)
5′-CACTGTGGTTACAACAGAGGT-3′.
right end primer sequence:
(SEQ ID NO. 5)
5′-TCTCTTCTCGTTGCTGCTCA-3′;

### (2) H79 Marker Primers:

left end primer sequence:
(SEQ ID NO. 6)
5′-CCGTGAGTTCACTTGTAA-3′,
right end primer sequence:
(SEQ ID NO. 7)
5′-GTACGATTTGACCAGCGAG-3′;

### (3) 33-3 Marker Primers:

left end primer sequence:
(SEQ ID NO. 8)
5′-CCTCGCTTACGAAATTAGTG-3′;
right end primer sequence,
(SEQ ID NO. 9)
5′-TGTGGCAGAGCAAGAGGG-3′;

### (4) 33-4 Marker Primers,

left end primer sequence,
(SEQ ID NO. 10)
5′-GCTTTCGGATTATCTTCACTA-3′,
right end primer sequence,
(SEQ ID NO. 11)
5′-AGCTCCCTAAGCTCAACCA-3′.

The nucleotide sequence of the molecular markers H99, H79, 33-3, 33-4 are respectively shown in SEQ ID NO.12-15.

The present disclosure also provides a molecular marking method for detecting whether the brown planthopper resistance gene BPH33.2 exist in rice, comprising:

amplifying genomic DNAs of rices to be examined by the marker primers, and detecting amplification products; if a DNA fragment of 96 bp can be amplified by H99 marker primers, or a DNA fragment of 120 bp can be amplified by H79 marker primers, or a DNA fragment of 330 bp can be amplified by 33-3 marker primers, or a DNA fragment of 868 bp can be amplified by 33-4 marker primers, all of them indicate a presence of a brown planthopper resistance locus BPH33.2 in rice insect-resistant varieties; if only a 112 bp DNA fragment can be amplified with H99 marker primers, or only a 129 bp DNA fragment can be amplified with H79 marker primers, or no DNA fragment can be amplified with 33-3 marker primers, or no DNA fragment can be amplified with 33-4 marker primers, all of them indicate an absence of a brown planthopper resistance locus BPH33.2 in rice insect-resistant varieties.

The present disclosure also provides a recombinant vector, wherein the recombinant vector comprises the rice brown planthopper resistance gene BPH33.2. The recombinant vector can be obtained by conventional methods in the art.

The present disclosure also provides a recombinant bacterium, wherein the recombinant bacterium comprises the rice brown planthopper resistance gene BPH33.2. The recombinant bacterium can be obtained by conventional methods in the art.

The present disclosure also provides an expression cassette, wherein the expression cassette comprises the rice brown planthopper resistance gene BPH33.2. The expression cassette can be obtained by conventional methods in the art.

The present disclosure also provides a transgenic cell line, wherein the transgenic cell line comprises the rice brown planthopper resistance gene BPH33.2. The transgenic cell line can be obtained by conventional methods in the art.

The present disclosure also provides an application of the rice brown planthopper resistance gene BPH33.2 in improving rice resistance to brown planthopper.

The present disclosure also provides a method of preparing transgenic plants with brown planthopper resistance, comprising:
transferring an expression cassette containing the nucleotide sequence shown in SEQ ID NO.1 or SEQ ID NO.2 into an insect-susceptible plant variety to obtain transgenic rice with brown planthopper resistance.

The present disclosure provides a method of breeding plants with brown planthopper resistance using transgenic technology, comprising:
1) transforming polynucleotides containing the gene BPH33.2 into cells of plant callus; said polynucleotide sequence being as shown in SEQ ID NO. 1 or SEQ ID NO. 2;
2) regenerating the transformed plant cells into plants;
3) culturing the regenerated plants and allowing expression of said polynucleotides, and harvesting T1 generation seeds;
4) sowing T1 generation seeds, detecting the gene BPH33.2 using molecular markers primer 33-3 and 33-4 as described above, and harvesting genetically pure T2 generation seeds.

The present disclosure also provides a method of generating plants with brown planthopper resistance using conventional breeding (non-transgenic), said method comprising crossing a plant having the brown planthopper resistance gene BPH33.2 with other plant to generate a progeny plant with brown planthopper resistance. Wherein said plant is monocotyledonous plant such as rice.

The present disclosure has the following advantages:

(1) The present disclosure successfully cloned a rice brown planthopper resistance gene, BPH33.2, using the mapping cloning method in forward genetics, and verified its function of brown planthopper resistance in rice.

(2) the gene BPH33.2 of the present disclosure is a completely dominant insect-resistant gene, and there is no significant difference between gene purity or heterozygosity for brown planthopper resistance, so that it can be better applied in the breeding of hybrid rice for insect resistance.

(3) the gene BPH33.2 has strong resistance to brown planthopper at the seedling, tillering and adult stages of rice, and has good resistance to several brown planthopper biotypes and infestation-causing types, as well as white backed planthopper, which is of great significance in breeding for insect resistance.

(4) The molecular markers in the present disclosure that are closely linked or co-segregated with resistance genes are highly reliable and easy to identify. As long as these molecular markers are detected by simple experiments, the presence or absence of brown planthopper resistance in the rice plant can be accurately predicted, and then its brown planthopper resistance can be predicted, independent of the influence of the environment, and the detection is convenient, fast and efficient.

(5) The present disclosure can improve the efficiency of auxiliary selection in conventional breeding and save costs. Through molecular marker detection of brown planthopper resistance gene loci in rice plants, highly resistant single plants can be rapidly identified only at the seedling stage, and susceptible single plants can be eliminated in a timely manner, which not only saves production costs, but also improves the efficiency of selection of resistant materials and shortens the breeding cycle of rice varieties.

### Embodiment 1: Fine Positioning and Clonal Validation of BPH33.2

### 1 Near Isogenic Line Construction and Effect Evaluation of BPH 33

The development of molecular markers linked to the gene BPH33.2 was referred to the RiceVarMap database of Huazhong Agricultural University (http://ricevarmap.ncpgr.cn/). Firstly, all the variant information of InDel (insertion deletion) in 0-1 M interval of chromosome 4 was found in the database, and then the variant sites with InDel of 3˜30 bp were screened out, and then the specific primers were designed at a distance of 50˜60 bp on both sides of this segment, so that the size of the amplified target fragment containing this deletion was around 100 bp. Primer length of 18˜20 bp is appropriate, and GC content is generally 40˜60%, and the selected primers are blasted on the database to eliminate primers with low specificity. Then the primers that were evenly distributed in the 0-1 M interval were selected for DNA fragment amplification among the resistant parents, and the PCR products were separated on 4% polyacrylamide gel for silver staining, and the colour was developed in 0.5 mol/L NaOH-formaldehyde solution, and the banding pattern was photographed on a photogelator for storage. The primers with obvious differences in band patterns (upper and lower differences, no common bands) between the resistant and susceptible parents were considered as the primers of candidate molecular markers. H99 and H79 were the molecular markers screened to be closely interlocked on both sides of BPH33.2.

### H99 Marker Primers:

left end primer:
5′-CACTGTGGGTTACAACAGAGGT-3′,
right end primer:
5′-TCTCTTCTCGTTGCTGCTCA-3′;

### H79 Marker Primers:

left end primer sequence,
5′-CCGTGAGTTCACTTGTAA-3′,
right end primer sequence,
5′-GTACGATTTGACCAGCGAG-3′.

The nucleotide sequence of the molecular markers H99 and H79 are respectively shown in SEQ ID NO.12-13.

In order to finely position the gene BPH33, and also to construct a molecular marker-assisted selection system for the gene BPH33 to be applied to insect-resistant breeding, the applicant constructed a near isogenic line of BPH33 in the context of the 9311 rice variety. The specific steps were, firstly, to use the molecular markers H99 and H79 on both sides of BPH33 to select a single plant with pure insect-resistant parental genotype at the BPH33 locus in the BC₁F₃ family line (crossing the insect-resistant parent KOLAYAL with the insect-susceptible parent 9311, and the F₁ obtained was backcrossed with 9311 once to obtain BC₁F₁, and then two consecutive selfings were performed to obtain BC₁F₃), and then to backcross with 9311 to obtain BC₂F₁, and then to select a single plant with pure insect-resistant parental genotype at the BPH33 locus in the BC₂F₁ family line. BC₂F₁ was obtained, then BC₂F₁ heterozygous for the BPH33 locus was further backcrossed with 9311 to obtain BC₃F₁, and a single strain heterozygous for the BPH33 locus was selected for self-crossing to obtain BC₃F₂, and a single strain with the pure resistant parental genotype at the BPH33 locus was selected for self-crossing to obtain BC₃F₃, which is the near isogenic line (NIL-BPH33) of BPH33 The near-allele line of BPH33 (NIL-BPH33) was obtained.

Seedling resistance and brown planthopper honeydew area were determined for the near-allelic lines and the parents, respectively. The modified seedling group identification method was used as the main method for the identification of brown planthopper resistance in this example. The susceptible control, resistant control and the material to be identified were sown at the same time. Each material was sown with 20 full seeds in a separate black plastic seedling box (7.2 cm calibre, 5 cm bottom diameter, 8 cm height), and when the plants reached the three-leaf stage, 12 uniformly growing seedlings were retained in each row, and the trays were placed in 200-mesh screens. The number of second instar brown planthopper was accessed at 8-10 heads per plant. When more than 90% of the plants in the susceptible control were dead, the resistance level could be evaluated according to the degree of damage of each seedling, and the resistance score value of each material was calculated by weighted average. Honeydew area test: Four-leaf stage rice plants of uniform growth were selected and transplanted into plastic cups (9 cm calibre, 500 ml), one seedling in each cup, and incubated with the prepared nutrient solution. The mouth of the cup was sealed with a round filter paper, then a small hole was drilled in the center of the filter paper and the rice stalks were stuck into the filter paper with the hole in the center, the cup with the brown planthopper larvae was put over the stalks, and then the same plastic cup was inverted on top. After 48 h of feeding, the filter paper was removed, a number was written on the edge of the paper with a pencil, and the paper was placed in an oven (50° C.) for 20 min. After drying, the paper was taken out and evenly sprayed with 0.25% ninhydrin/ethanol solution, and then placed in the oven at 60° C. for 30 min to develop the colour, and the purple part of the filter paper was the area of honeydew secreted by brown planthopper. It was photographed and scanned, and the area of staining was calculated on imageJ software.

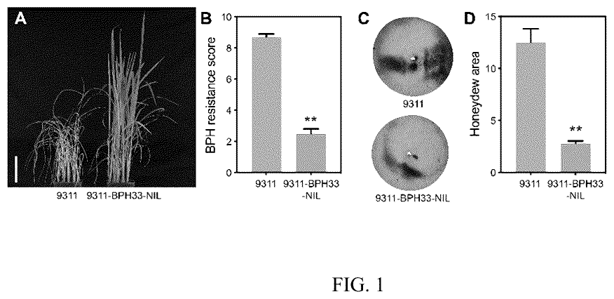

The results are shown in FIG. 1. It can be seen that 9311 showed susceptibility to brown planthopper and NIL-BPH33 showed resistance, and the area of honeydew secreted by brown planthopper on 9311 was obviously more than that of NIL-BPH33.

### 2 Fine Positioning of BPH33

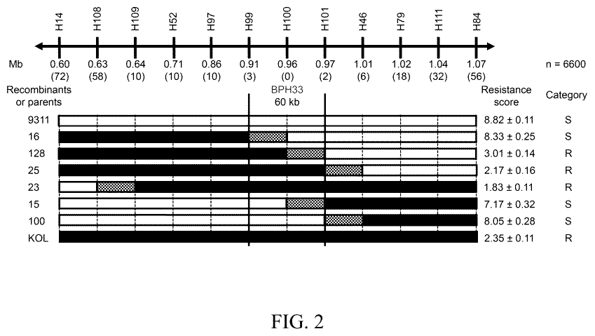

In order to finely position the gene BPH33, the applicant selected BC₃F₁ lines that were genotypically heterozygous for both the molecular markers H14 and H84 (for the methods for obtaining BC₃F₁ lines, see the Hu et al. 2018 Rice 11:55), and self-crossed them to obtain a BC₃F₂ segregating population containing 6,600 individual plants. Based on the results of the preliminary positioning, all the single plants were genotyped using closely linked markers on both sides of the target gene fragments, and the single plants with recombination exchanges between the two molecular markers were selected (i.e., pure 9311 genotype B at locus H14 and heterozygous genotype H at locus H84; or heterozygous genotype H at locus H14 and pure 9311 genotype B at locus H84, etc.), and those that had undergone The single plants in which recombination exchanges occurred were transplanted to the field for planting, and at maturity, the single plants were collected as self-crosses and used for subsequent genotypic and phenotypic analyses. Molecular markers that were uniformly distributed and polymorphic between the two parents were developed in the target gene regions, and genotypic analyses of these recombinant individual plants were continued to construct a physical genetic map of the target regions based on the physical location of the molecular markers on the chromosomes (FIG. 2). One to two individual plants of the same exchange type were selected for progeny testing. BPH33 was dominant in this example, and for the HA and AH exchange types, we could not accurately determine which genotype provided the phenotypic effect. HA refers to the recombination exchange on both sides of the marker, the molecular marker genotype on one side is the heterozygous genotype, and the insect resistant parent kolayal is the homozygous genotype on the other side. AH is a recombination exchange on both sides of the marker, one side of the marker genotype is the homozygous genotype of the insect resistant parent kolayal, and the other side is the heterozygous genotype. Therefore, for the identification of brown planthopper seedling resistance in recombinant individual plants, we took a progeny test. The recombinant individual plants were collected and numbered, and 1˜2 numbers of the same exchange type were selected and sown in 96-well PCR plates, and about 50 seeds were sown in each number, and then genotyped with molecular markers in the target region, and the individual plants of the AB and BA types were selected, and each line could be selected with about 8˜12 individual plants, which were planted in rows, and then seedling resistance identification was carried out, and the seedling resistance was determined according to the mean resistance scores of selected The phenotypes of the recombinant individual plants were then determined based on the average of the resistance scores of the selected progeny. Combining the genotypes and phenotypes of the recombinant monocultures, BPH33 was finely positioned between markers H99 and H101, corresponding to the reference sequence Nihonkaru with an interval of 60 kb (FIG. 2).

### 3 Candidate Gene Analysis of BPH33

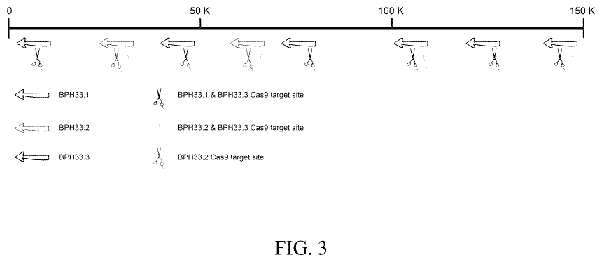

In order to find out what candidate genes are inside the BPH33 fine-positioned interval, the applicant sequenced three generations of the genome of the parent KOLAYAL carrying BPH33. It was found that the 60 kb interval of the reference sequence Nipponbare is 160 kb long in KOLAYAL, which is 100 kb longer, and among these 160 kb, gene prediction using FGENESH revealed a total of four genes, the first of which is very similar to Nipponbare and encodes a C₂H₂ zinc-finger-like transcription factor. The latter three genes, all encoding a class of atypical LRR proteins and presenting multiple copies, were structurally different from the reference genome, and were therefore identified as reliable candidates for BPH33, named BPH33.1, BPH33.2, and BPH33.3, which were present in six, two, and four copies, respectively (FIG. 3).

### 4 Function Validation of Candidate Genes for BPH33

In order to figure out which one of BPH33.1, BPH33.2 and BPH33.3 is BPH33 or all of them are BPH33, the applicant conducted transgenic validation using gene knockout and genetic complementation experiments respectively.

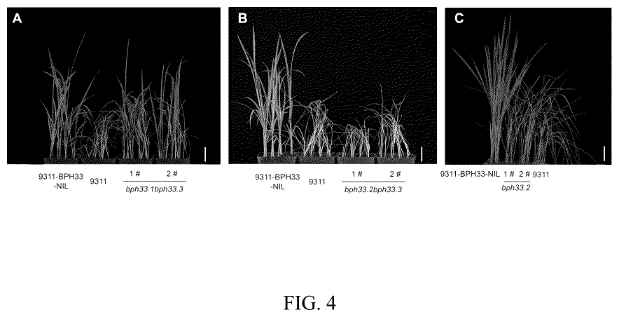

CRISPR-Cas9 was first used to knock out both BPH33.1 and BPH33.3, as well as BPH33.2 and BPH33.3 in the insect-resistant BPH33 near-allele line. It was found that most of the BPH33.1/BPH33.3 pure mutants were insect-resistant, and did not differ from the near-allele line in terms of resistance; whereas all of the BPH33.2/BPH33.3 pure mutants were insect-susceptible and significantly different from the near-allelic lines for resistance (FIG. 4). Further analysis revealed that all BPH33.1/BPH33.3 double mutants phenotyped as insect-susceptible had a large deletion between the genes BPH33.1 and BPH33.3, i.e., the gene BPH33.2 was also deleted, and they were actually BPH33.1/BPH33.2/BPH33.3 triple mutants (FIG. 3). These results indirectly indicated that neither BPH33.1 nor BPH33.3 was BPH33, whereas BPH33.2 was most likely BPH33. The applicant further designed a specific target on the second exon of BPH33.2, and obtained a pure single knockout mutant of BPH33.2. Resistance identification results revealed that all BPH33.2 mutants were susceptible (FIG. 4), thus confirming that BPH33.2 is BPH33.

### Embodiment 2: Development and Validation of the BPH33.2 Molecular Markers

### 1 BPH33.2 Molecular Markers Development and Identification

Based on the CDS sequence of the cloned BPH33.2, the sequence of BPH33.2 was multiplexed with the sequences of two other homologous genes, BPH33.1 and BPH33.3, as well as the sequences of a number of the third generation sequenced varieties (R498, 9311, Zhenshan 97 (ZS97), Minghui 63 (MH63), and Nipponbare (NPB)), and the sequence of the cloned brown planthopper resistance gene, BPH30, in the same region, and another cloned brown planthopper resistance gene in this region, BPH30, were subjected to sequence multiple alignment. Then primers were designed in the BPH33.2-specific region, and two pairs of primers, 33-3 and 33-4, were obtained. Wherever the target fragment could be amplified by PCR using these two pairs of primers, the presence of the functional allele of BPH33.2 was demonstrated, whereas the absence of the target fragment indicated that the allele was not carried.

The sequences of the primers are as follows:

### 33-3 Maker Primer:

left end primer sequence,
5′-CCTCGCTTACGAAATTAGTG-3′,
right end primer sequence,
5′-TGTGGCAGAGCAAGAGGG-3′;

### 33-4 Marker Primers:

left end primer:
5′-GCTTTCGGATTATCTTCACTA-3′,
right end primer:
5′-AGCTCCCTAAGCTCAACCA-3′.

The nucleotide sequence of the molecular markers 33-3 and 33-4 are respectively shown in SEQ ID NO.14-15.

### 2 Validation of the BPH33.2 Molecular Markers

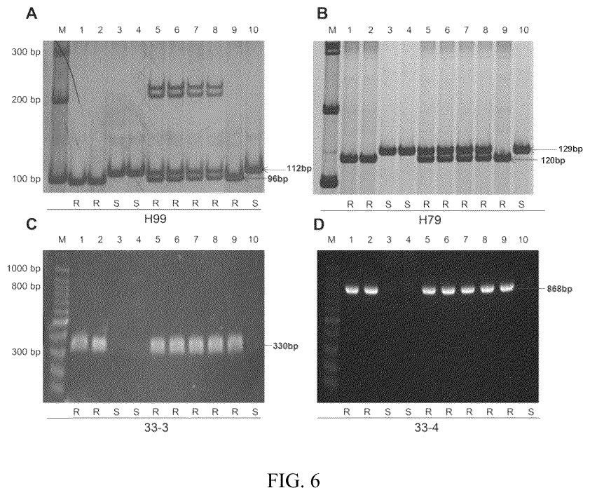

The materials with bph33.2 gene locus as heterozygote in BC₃F₂ generation (the BC₃F₂ generation was obtained in a same way sa in Embodiment 1) were selected, and 92 individual plants were planted, and then 92 materials harvested from these 92 individual plants were BC₃F_{2:3} segregating population. To validate the efficiency of BPH33.2 molecular markers in screening for functional alleles of BPH33.2, we genotyped 92 lines from a BC₃F_{2:3} segregating population of 9311/KOLIYAL carrying BPH33.2 for molecular markers, which were then combined with seedling resistance phenotypes of each line, and validated the markers H99 by t-tests and association analyses, H79, 33-3 and 33-4 were co-segregated with brown planthopper resistance phenotypes (FIG. 6).

### Embodiment 3: Molecular Marker-Assisted Selection of Improved Brown Planthopper-Resistant Rice Lines Carrying a Functional Allele of BPH33.2

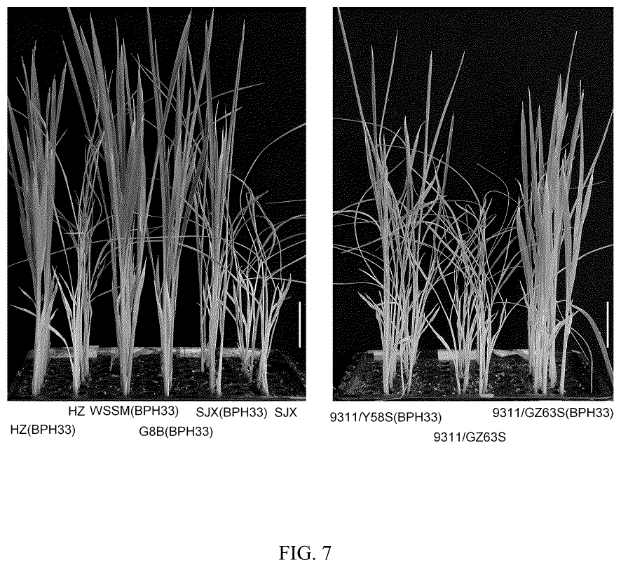

Using molecular marker-assisted selection (MAS), combined with conventional backcross breeding technology, the BPH33.2 functional allele was introduced into excellent insect-susceptible rice varieties (9311, Huanghuazhan, Huazhan, Wushansimiao, 19xiang, Guang 8B), and a series of rice lines with markedly improved brown planthopper resistance and excellent agronomic traits were obtained, which can be used as intermediate materials for the breeding of new brown planthopper-resistant rice varieties. The material can be used as an intermediate material for breeding new brown planthopper resistant rice varieties. The specific method of operation is to cross the improved varieties with the parent KOLAYAL, which carries BPH33.2, as the rotating parent to obtain F1 hybrids. Then the rotating parent was crossed with F1 to obtain BC₁F₁, and a single plant heterozygous for BPH33.2 was selected by MAS to be crossed with the rotating parent to obtain BC₂F₁. BC₂F₁ heterozygous for BPH33.2 was then selected by MAS to be crossed with the rotating parent to obtain BC₃F₁, and a single plant of BC₃F₁ heterozygous for BPH33.2 with the closest agronomic traits to the rotating parent was selected to be crossed to obtain BC₃F₂, and a single plant of BC₃F₁ heterozygous for BPH33.2 was selected by MAS to be selfed to obtain BC₃F₂. BC₃F₂ was obtained. BC₃F₃ was obtained by self-crossing a single plant of BPH33.2 as pure KOLAYAL genotype with excellent agronomic traits using MAS. BC₃F₃ was obtained by self-crossing a single plant of BC₃F₃ with excellent agronomic traits. BC₃F₄ was obtained by self-crossing the single plant of BC₃F₃ with good agronomic traits. The results of brown planthopper seedling resistance identification showed that the improved line was significantly more resistant to the insect relative to the control that did not carry the gene BPH33.2 (FIG. 7A).

In addition, improved lines of 9311 (carrying BPH33.2) were also used to cross two sterile lines, Guangzhan 63S and Y58S, respectively, to obtain a two-line hybrid rice heterozygous for BPH33.2. The results of insect resistance identification showed that the improved two-line hybrid rice had significantly improved brown planthopper resistance compared to the control (FIG. 7B). These results indicate that BPH33.2 functions independently of the genetic background of rice and is a fully dominant gene, which is promising for breeding insect-resistant hybrid rice.

### Embodiment 4

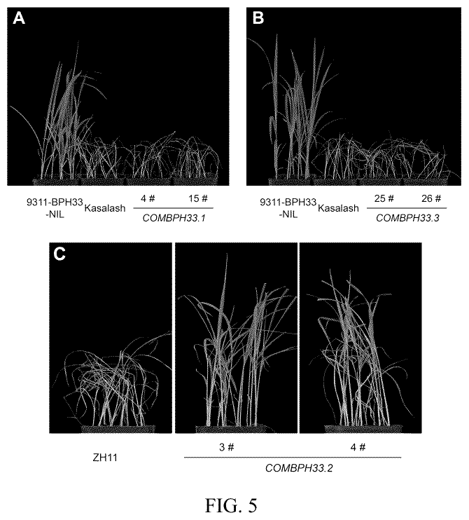

The applicant cloned the genomic sequences of BPH33.1, BPH33.2 and BPH33.3 in the BPH33 insect-resistant near isogenic line (promoter+5′UTR+CDS+3′UTR) and ligated them into PCAMBIA1300 vector respectively, which is the recombinant candidate gene expression vector. The three vectors were then transformed into the insect-susceptible rice varieties Kasalath and Zhonghua 11 by Agrobacterium-mediated genetic transformation, respectively. The transgenic pure T2 generation lines were identified for brown planthopper seedling resistance, and the results showed that the transgenic lines of both BPH33.1 and BPH33.3 exhibited insect susceptibility, while the transgenic line of BPH33.2 was insect resistant (FIG. 5), and these results further demonstrated that BPH33.2 was BPH33. The nucleotide sequence of BPH33.2 is shown in SEQ ID NO. 1, its cDNA sequence is shown in SEQ ID NO.2, and its amino acid sequence of a protein it encodes is shown in SEQ ID NO.3.

The sequences are as follows:

BPH33.2 gene
(SEQ ID NO.1)
GAATTCACCTTCCGCGCACGCAAAATGGAGTGATGTATTTTCTTACGATTAA
TTAGGTATTTACTATTTTTTTGAAAAATAGATTAATATGATTCTTTAAAGCAA
CTTTCGTATAGAAACTTTTTGCAAAATACATATTGTTTAGCGGTTTGAAAAG
CGTACACGCGGAACACGACGGAGGTGAGTTGGGAAAAATGGGGGAAGAA
CACAACCTAAGTCTTAAACTCTCCTCCACGGCTCCTCTATCGAGAACCACT
CATTCTCTCCCTCTCCTATCTTCTCTCTCTCACTAGATCCACACACGAGCTCT
CCTTATCCTTCACTTGCCACCGTTTCTCCTCGTGTAGAAGCAACCACACGG
CTTGCCTTGGCTTGGCGACGGCGACTTCGAACACATGGCATTGGATGAGCA
GTGAAGGGGAAGGGGTGGGTCGGCATTATCGAACGAGGGCCAGAGCTTGG
GACTAGCGACAACAATGGCGACTGCGGCAAGGGCAGGGTGGTGGTGGCA
AACAATGGGCTGAGCTCAGGATCTGTGGTGAACGAACATCTATGTGGGTTG
TTTACAACAATGTTGTGGCCCTAGATCTGTTGGAGTGAAGGCTAGATCTACC
ATCGAGGAGGTCAGATCTGGATGGTGGCTATTACCTCTCTCGATGCCGCCG
CTAGTGGGCAGATCCAAGCCATCATGCTTCATGTCTCCTGCGGCGGGTGGC
GGTAGATCGGGGTACTGGGATGGTGGTTCACTACTAGAGAAATGCACTTCG
GTGCCTGTTGGGAAGACCCCTTAGATGTCGGTTTTCCTAACCAGCACCATG
AAGGCCGCACTGATTACCGGCACCAATGTCATTTTAAAAACCGACACCAAT
ATGGAATCAACGAGCAAAAGAAAAAAAAGCCAGCCGGCTCTAGCCGATTA
ACTCCCACATGAACCCACATGAACAACAAACAAATGATGAACAGATTCCA
CATCAACAGATGAACAAGAGCGACACAAAATCTCACACCAATTATCATAGC
AACAAATGAACTCTTGCATGAACAAGAACGTATCTCAAACAAATCACAAA
GTGGGAGGGAGGGGGAGAGATGGATCCACTGGCCGCCGATGCCGTCACAC
CTCCAGATCCAACACGGGCCACCTCCGTCCCGCCACCGCACGCCGCCTCCC
GCCGCCAAGGGGCGCAGACCGCCTCCGTCCCGTCGCCGCACGCTGCCGCA
CTTCGCCTACCACCGCCGAGGGGCGCGGACCACCTCCGTCCTGCCGCCGCA
TGCCGCCTCCGATCGCCGAGGGGCACGGGCTGCCTCCTTCCCGCCGCCGC
ACACCACCGATCTCGCCGCCGAGGGGCATGCAAGACGACGGTGAGAAGGC
TAGATCCAGCTGCTGGGACCCTGTGAACTCCGGTGGTGGTGGAACCTCCCT
GGCTTCTTAGGGACAGCGACAACAACTTCAACGTCGACCACATGATTTTCG
CATGCCTTATACTTGTAAGCGGGGCGGCTGGGCGCCCATGAAAAATCATTTT
TTTCTACCTGCGAATACCCATCTTGTAGTGGTGCTAGATTAGCAAACCTGTT
TTCTCCTTCTTTTTTTTGGGGGGGGGGGGTGTTTGAACTATGGTGGCTTAGG
TAGCCTACATGCCAATCAAAGTTTCAAATTTAGTTTAACTCCAAGGGAGAAT
GTATTCTATCGAATTTAGAAAAAATGTCAAAATTCATGATTTATTTAATTGTT
TGTGGAATTATGTTTGAAATTTTGTAATTTATGTTACCCCGAAATGTTTATGC
ATTATGAGATAGTTAACCCTATACTCCCTCCGGTTCTATATTAATTGACGTTTT
GAATAAGGTTGAGGCTAAACTTTTATAACTTTGACCATCAATAACTTTAAAA
ATATTTAATTTAAAGAAAATAGAATAACATATATAAATTTTTCTTTCAAAGCA
CTATAATAAAAGTAAACATGCATTTATTTATTGTATATATTATAATAGAAAAAT
AAGATCAAAGATATATCTTGTAGACTGTGTCATTGTCCAAAACGTCAATTAA
AATGATACTGGAGGGAGGGAGTATAGGGTATCATATTCTAACCTTGGGGTTA
ACAAGACACAGTTGCTTTTGTATCCTGGATTGACGACGTACATCCCTTTTGC
TTTATCTTTTGCTTTTTGCACTTTAATCAAGGAGGCGAAGCAAAGAATACGA
CGATTAGCCAACTACTAACTCCAAATTATCTATAGCCAATTTAATAGTTGATT
TATACAACAATTGCTTACAATACTATTAATATGTGATCCCACCTGTCATAGAA
GCATATGTTATGTCTTGAAGTCCGTGTTACAGCTGTCTACAGATCTGTAGCC
CACTGCTTTTCTCTCTACTTTTTTATCTCTTTAAAATATATATGTTTATAGCTG
GCTTATAGTCTGCTATTGTACCTCCTCTCAGATGATGCATTCAGGTACAGCTT
AGGACGTACATATAGCATATCGATTTGCGGCTTTGCAGCAAATGCAAAGCA
AAGAAAAATACTAAAGCAAACATATCGATCGTACGCAAGGAATAATAGATC
GACGAAGCATGTTGTAATAATGCAAGCATTCTAGACGACAGTACGTTTTTAC
AAGGCTTCTGCTAGCTCTAGCCCAGAACATCGATCTATCGATCATATTGCAT
CCAGTTTGATCGTTTCAATTTATGCTCGCCCTCGCACCAACAATGGCTTAGC
TAATCCCCATGTACTCGATCTCTGTCGGCAAACATCGTGATTAAGGTAAACT
AGCTACATCATATCGTGCAGTTTAATTGCGCTCTCTCTCTCTCTCTCTCCCCC
TATGTGTGTGTGTTGATGCATCAACATGCATATATATATATATATATATATATATA
TATATATATATATATATATATATATATATATATATATATATATATATATATATATATATA
TATATATATATATATATATATATATATATATATATATGTATGTATGTACAAAATCAT
CTCAGTTTTATAAGCTTTAATTTTAGTTAGATCTCTAGCTATGTGGTATATTAT
TTAATTTGGTTTTCTACGGGATGTTCACACATATATAAATATATGAAATGCTT
GCATGCTGTTGTAATTCTGGCTTAATTTGTACGTTTCGTTTATATGAAAAGAT
GATTTAGTTTATATAACATGCATGTAAACACTTCTCCATTATCCTCAAAACAA
TCCCTAGGGAGAACGAATTAAAGATATATATGCATCCGTGTAGTATATCATTT
TCGTCGTTGATTATTAATCAAATTAAAATACACTTGCATTCACTTGTGTCCAT
TTGAAACCCAGGCGCATGTTGGGCCGGGCCACTAGTACAAAATTAATCATG
TACAGTAACTAATTTATTAAGTATATATGTTTTACATGCGTATATACATGAACT
ACTGTATCTATAATGTGCAGGCTGGAGCGAGTAATTAAATAATCAGGAGTGG
GCTGGAGAAATCGATCCATCAGGCATCTATATATCATGGGTTAATTAATTACA
GGGCTTGCTTCAACATATATATGTATTCTCTGCTGTCGATCCATTTCTAATTAA
GGAGCGTTTAATCAACTGAAAGAAGATCGATTGAGATTATATGATCGATGAA
GCTGCCCGCTTAACCGCAATTCAACATTAATTGAAGAGAGCCAGGCTACGC
AGATCAACTCAACATGCCACCAGCGGAGGTAATTAAATCAGATCGGTGATA
TTGTTTTTATTTTTGAATGAAAGTTATTTCACGACCACGCACGTGAATTAAA
CAAGGAAAACTTTACTGCTATTTTAGATTTCCAATTTGGACGCAATTTTTTG
AAACACGCTTTCATTGTCATATTTGGCTTTAGAGGTGTTTTTTTTTCTAACAT
CTCATTTGCAAACAATCCTCTCGGAGCAAACTTCTTCTATTTGTGGAAGAAA
TAAATTGCAAAAAGGATAATTTTACCATCCTTAGAAGATAACATGAGGTACC
ATATTTTATACTATAAATTTTGGCACATCAAGTATTATGTATCTCAATGTATAA
AAGAAATAATTTTAGTGTAAACTTTTGATAATAACAAGTAATTGGTCAAGAA
CCGTAAAATTTCTCACACTCCACCTGTTCAAAATTCAAATGCTATGTTTGGC
TAGTTGCAGAGAGAAAAAATATATATAGTGTAATTAATAACTTTATTCGTAAC
ATTATATATATGCCGGTTTGCACTCTACCTTTTTGTGGTCTATTACACTTTATT
CTATTGTAATTAATAAATACACCACCGAGAAAACTGGGCGTTTCTTTAGAAT
TTTATTTGTGATAAAAGATTTTAGTGTGTTTGTTTAGCTATGAACTTGTTTCA
TTGTGACAGAGGGAATAAATATACTAATGAAAATAATTAAAACTAGCTAATT
CAGCTTTGCCTGTATTGATCGCGGAATCACCGACCGAGAAGCTCTCTAGTT
CTAGGGCAGGTTTGAATTAATTAGTGGTATTAATGAATGATTAATTATTGCAG
TGGACCATGATCATAGCAGACAGCATCGATGAAGCAGCAGGCTGGGTAATT
GAATTTCTGGAGGATACAAGCAAGGGGAATGTGATGTATTTCCATGGCTGG
CATGGACTGGGGGCTTCAGCCGTCCTCAGAGCAGTCGCCAAAAGACTGAC
GATGAGGCCATCGCCATCGCAAGGAAGAAGAAGGTGGGAGAAGATCATCC
ACGTTGATTGCTCTCTGTGGCAGAGCAAGAGGGCCCTGCAGAAGGCCATC
GCCCAGGAGCTGCAGCTTCCTCGGTCGCTGATGGCCTTGTTCGACCAGCAC
GACGAGGAGGATGATTTCAGTGGGGTGGATCAGGGCATGAGAGGGGTGGT
TCCACTTGTCACACAGGCAATCTTGAGCGACCTGGTAAACCACACATTCCT
GGTGATCTTCCACAATGGGAGCGACAGCTACATTGATCTACAGGAGTGTGG
TGTCCCTGTAATAACGGGACTCTTGAACAAAACGGTGTTGTGGACCTCCCG
AAGCAGCTTTCGGATTATCTTCACTAATTTCGTAAGCGAGGATCGGCATAAA
CTTGCTGGGCTGAGTGATGCCGCTATATTTGCTGATTCTATTGGAAACATATT
GGGAATGCTTTTGCATCAAGAGGCAGAGGAGGTTGCCAGGTACACCGGTG
TCCCTCAATCTGCTGGCATGAGCACCGAGCTTGTCAAGAAGTGCATCATGT
ATCAGTTGATGCTAAGACAACAACATGTGGACTACACCCAACACTGGGATA
CCCACGCAGGTAACTATTGGGTATGTAGTGGAATCATACAAACCTCTTCAGA
CACCACTAGTAGTACAAGTTGTCATAGCTCATCGCCATGGGAGATAGCCCA
AGCTCTTTATAATAACTTGATCTTGGAGTTTTTGACCAGGGACAATTACAAC
ATGAAACTAATTTCTGTGGAAGAAATACAGAGTACCCTACGACTGCCTTCT
GATTTTGTGGACGAGTCGTCCTTCTTCCGGACATGTGATGCTGCTGGCAATA
ACAATGTGGACGCAACTACAGCTTGTTGTAGACAGAAATCACTGGAGGCC
AAAATGTTTCAACATCCATCTGCACGCAGTTTGCGTGTGATACATCTCTTCG
ATTGTACCTTCAGTTTTGCATCTCCACCCTTCCTCTCTTGCAGCAGCCTAAG
ATTCCTCCTACTTGACCATTGCAAAGACAAACACAACCTCGGCTCAGCACC
AAATAGTACTAGTGCTGGAGACACTGAAAAGGAGACTAGTATTAGTAGTGG
AGCATGTTTCCAGAAGCTATGGGTGCTTGACCTGAGTTACACAGATTGGTAT
TGGCTGCTATCAGTAGAGGCACAAGATCTGATGGTTGAGCTTAGGGAGCTA
AATGTGAAGGGGGTCAAGCATTGGAGCATAAGCCATCTACTCCGTGATGAC
AACAATTCTAGTACTGGTGTCGGAAGCAGCACCAAGCCCCTTGGGCTACTC
AACCTTGTCAAGCTCCAAGTCACAACCGAACCAATAACTGAAGATCAACAT
CAGTCACAAGTATGGAAGGAAGATCAGGTAGCAGCAACATTATTCCCGAAC
CTATCTAGCTGCAAAATCGTAAAGACGATCATTCTTGATGGTTGTTTTGAGT
TGAAGCGAATTGACCCTCACGTCCTGCCACCATCACTCGAGTCATTTAGCT
TCTCCAGCAGCAGCAATGACAATGATGTTCATGTCTCGGCCAAGATAGAGA
GCATCTCCTTCCGGGGATGCACCCAGTTGAAGAGTGTGCTTTTGAGAGGAC
TATTTCTAAGGCTCAAGCAGCTAGACGTGTCAGAAACTTGTATTAAAACCC
TCGACCTACGTGCAATGCGAGGCAACGGGTCTCTCAAGGAGCTATTTCTGC
TTGGATGCAAGGAGCTTCGTGCAATACTATGGCCAAAACAAGATGTATCATT
GGAGGTTCTGCACATCGACACTAGTAGCACAGAACTTGATCATGCTACAGG
TGTAGAAGAATCATCATCATTCTCACCTGTTGAATTTAAATGGTATATTTCAG
TGAGGGACAGAAGGCTCCTTCGCTCGCTAAATGATACAAAATATCCCTCGG
ATGCACCATGCATCGAGATCTCATCCCCTCCTGCTAGTGTTGCTACTGCTAC
TACTGATGGTTCTGAATTAGGCGGAACAATAAGCAAGAGAAGGCCCATTGC
CGTTAGCAGGGCTGAACAACGTTGGTTGATGTCGACCAAAAGCCGACGAC
CAGCTGCTGACAACAAGAAGTTATATGCGGATGTCGACTCCACGATCCAGC
ACTTGCAACTACAAGCAACCATGAACGGCAACTGGATGTGGCCTTATAAAC
AGGAGGGCAGCACCTCTCACTACATTAGCTTACAAGATGATAAGAGGATGC
AGACGAAACCATTGTCGTCGCCATCTTTGCCAGGTTCCATCTGTGAAAGAG
CTTTAGGCCTGCACGTGCATGACAGCTTGTCTATCGCAAGTATCACAAGCCA
TTCAAATATGGCACGGAGATGGAATAACCTAGACTGGTGCAGAGTGGAGA
GATGCCCTAACATTGAAGGTGTTGTTTTCACTCCACCTAGTGATCCTGCTTG
TGATAAAATTTTCTGGTACCTGAAGACATTCTGGGCATCCCAACTAGCAAA
GGCGCAGCACATCTGGGGCTGGGGCACAACGGACCAGCTGCATTTTGAGC
CTGATGATAAGTCATTCTACCTGCAAGTGCTACACCTAGATTGCTGCCCCAG
GTTGATACATGTGCTTTCCTCATATGACAACAGTCCTTCCTATGCATACCGTT
GGTTGGAAACCCTTGAGATCGTGTGTTGTGGTGACCTCAAGGATGTCTTCC
GAGTGGATGATAATAATCAAGAGCTTCTAAAAACTATAGAATTCGAAGAAC
TGAAGCACATCCACCTGCACGAGCTGCCCTCTCTGCAACGCATCTGCGGGC
ATAGGATTGTGGCGCCCAAGCTCGAGACCATCAAAATCCGAGGCTGCTGGA
GCCTCACCCGCCTACCGGCTGTTGGTCTTGACAGTACCCGCAAGCCCAAGG
TGGATTGTGAGAAGGAATGGTGGGATGGCCTGGAGTGGGACGGGTTGGAG
AACGGACACCATCCTTCCCTCTTTGAGCCCACCCACTCACGCTACTACAAG
AAGAAACTGCCCAGAGGTTCCATGCTCAGGTATGTACATATGTCTGTGTGCA
TACATGATTGTTACTTGGGTATCATTCCTTGCGCGCACATACATACATACGTA
GAAACTTATATAGCTCAGTACTTTAGTTACTCCATCATATATATATATATATATA
TATATGATCGAGCTTTTCCCTTTTTTTTTAATTTTGTCTGACTTGGCGTGCTTT
CTTTTCTTCTTAAATTCAGGTGATCGATATATACGTCCATACACTACATATTTG
ATACTGTAATCATCTACGTATCATTCCCTTTCCCTCATCCAGTGGCTCATCAA
GTTCTTCAGCTCTATCTCTATTATATATGTCCTTATTTGGGGCTGGTGTTCAA
AGTGCTCGCTCATTTCATCATATCATTTGCAACGGCATCAATATCTAATGCAT
GATGAGTAAGTACATCTCCGGCCGGCATCCAGACGCTCGTCATCTGCTTGTT
ACTAGCTTGGATTCTGTTCATTTCTGCTTCTTTTGTTCTGTGTGAAAATTAAT
AAATGGATGCTCAGCTGCCAGCTGGTGTGTGAGCTCTGCATCTATCTGCTA
AGTTATTTTCTCCATACATGCTTGCTTTCCGTTTGGCCGGTTAGTCGATCTGC
TTGTTCGACCGATCCATCCATCGGATTACTACGTATGCTTCATCTTTCTGGAT
AGATGATCATACATGTATATGTGAAAACTTTGATCTCTAGCACCGTTCTGGT
GTGGTGTGCTTCCTAGAGGTCGACGAATAAATCATCAGCTTCAGCGGCATG
AGAAGATCCATTGGGTGATCCTCGTGCAGTCGAGTTGCTGTTGTGCACTTG
TTTGTGGGTGCGTGTGTGTTCGTGTGATTTGTCAGCATATGTGGACTGACCG
AGTTTGTACTCCTAATTACTTCACTTGTATGAACAGAAACAATGATGATGAT
TGTGAGTGTGACATATATTTTC
>BPH33.2 cDNA
(SEQ ID NO. 2)
AAATGCAAAGCAAAGAAAAATACTAAAGCAAACATATCGATCGTACGCAA
GGAATAATAGATCGACGAAGCATGTTGTAATAATGCAAGCATTCTAGACGAC
AGTACGTTTTTACAAGGCTTCTGCTAGCTCTAGCCCAGAACATCGATCTATC
GATCATATTGCATCCAGTTTGATCGTTTCAATTTATGCTCGCCCTCGCACCAA
CAATGGCTTAGCTAATCCCCATGTACTCGATCTCTGTCGGCAAACATCGTGA
TTAAGGCTGGAGCGAGTAATTAAATAATCAGGAGTGGGCTGGAGAAATCGA
TCCATCAGGCATCTATATATCATGGGTTAATTAATTACAGGGCTTGCTTCAAC
ATATATATGTATTCTCTGCTGTCGATCCATTTCTAATTAAGGAGCGTTTAATCA
ACTGAAAGAAGATCGATTGAGATTATATGATCGATGAAGCTGCCCGCTTAAC
CGCAATTCAACATTAATTGAAGAGAGCCAGGCTACGCAGATCAACTCAACA
TGCCACCAGCGGAGTGGACCATGATCATAGCAGACAGCATCGATGAAGCA
GCAGGCTGGGTAATTGAATTTCTGGAGGATACAAGCAAGGGGAATGTGATG
TATTTCCATGGCTGGCATGGACTGGGGGCTTCAGCCGTCCTCAGAGCAGTC
GCCAAAAGACTGACGATGAGGCCATCGCCATCGCAAGGAAGAAGAAGGT
GGGAGAAGATCATCCACGTTGATTGCTCTCTGTGGCAGAGCAAGAGGGCC
CTGCAGAAGGCCATCGCCCAGGAGCTGCAGCTTCCTCGGTCGCTGATGGC
CTTGTTCGACCAGCACGACGAGGAGGATGATTTCAGTGGGGTGGATCAGG
GCATGAGAGGGGTGGTTCCACTTGTCACACAGGCAATCTTGAGCGACCTG
GTAAACCACACATTCCTGGTGATCTTCCACAATGGGAGCGACAGCTACATT
GATCTACAGGAGTGTGGTGTCCCTGTAATAACGGGACTCTTGAACAAAACG
GTGTTGTGGACCTCCCGAAGCAGCTTTCGGATTATCTTCACTAATTTCGTAA
GCGAGGATCGGCATAAACTTGCTGGGCTGAGTGATGCCGCTATATTTGCTGA
TTCTATTGGAAACATATTGGGAATGCTTTTGCATCAAGAGGCAGAGGAGGT
TGCCAGGTACACCGGTGTCCCTCAATCTGCTGGCATGAGCACCGAGCTTGT
CAAGAAGTGCATCATGTATCAGTTGATGCTAAGACAACAACATGTGGACTA
CACCCAACACTGGGATACCCACGCAGGTAACTATTGGGTATGTAGTGGAAT
CATACAAACCTCTTCAGACACCACTAGTAGTACAAGTTGTCATAGCTCATCG
CCATGGGAGATAGCCCAAGCTCTTTATAATAACTTGATCTTGGAGTTTTTGA
CCAGGGACAATTACAACATGAAACTAATTTCTGTGGAAGAAATACAGAGTA
CCCTACGACTGCCTTCTGATTTTGTGGACGAGTCGTCCTTCTTCCGGACATG
TGATGCTGCTGGCAATAACAATGTGGACGCAACTACAGCTTGTTGTAGACA
GAAATCACTGGAGGCCAAAATGTTTCAACATCCATCTGCACGCAGTTTGCG
TGTGATACATCTCTTCGATTGTACCTTCAGTTTTGCATCTCCACCCTTCCTCT
CTTGCAGCAGCCTAAGATTCCTCCTACTTGACCATTGCAAAGACAAACACA
ACCTCGGCTCAGCACCAAATAGTACTAGTGCTGGAGACACTGAAAAGGAG
ACTAGTATTAGTAGTGGAGCATGTTTCCAGAAGCTATGGGTGCTTGACCTGA
GTTACACAGATTGGTATTGGCTGCTATCAGTAGAGGCACAAGATCTGATGGT
TGAGCTTAGGGAGCTAAATGTGAAGGGGGTCAAGCATTGGAGCATAAGCC
ATCTACTCCGTGATGACAACAATTCTAGTACTGGTGTCGGAAGCAGCACCA
AGCCCCTTGGGCTACTCAACCTTGTCAAGCTCCAAGTCACAACCGAACCAA
TAACTGAAGATCAACATCAGTCACAAGTATGGAAGGAAGATCAGGTAGCA
GCAACATTATTCCCGAACCTATCTAGCTGCAAAATCGTAAAGACGATCATTC
TTGATGGTTGTTTTGAGTTGAAGCGAATTGACCCTCACGTCCTGCCACCAT
CACTCGAGTCATTTAGCTTCTCCAGCAGCAGCAATGACAATGATGTTCATGT
CTCGGCCAAGATAGAGAGCATCTCCTTCCGGGGATGCACCCAGTTGAAGA
GTGTGCTTTTGAGAGGACTATTTCTAAGGCTCAAGCAGCTAGACGTGTCAG
AAACTTGTATTAAAACCCTCGACCTACGTGCAATGCGAGGCAACGGGTCTC
TCAAGGAGCTATTTCTGCTTGGATGCAAGGAGCTTCGTGCAATACTATGGCC
AAAACAAGATGTATCATTGGAGGTTCTGCACATCGACACTAGTAGCACAGA
ACTTGATCATGCTACAGGTGTAGAAGAATCATCATCATTCTCACCTGTTGAA
TTTAAATGGTATATTTCAGTGAGGGACAGAAGGCTCCTTCGCTCGCTAAATG
ATACAAAATATCCCTCGGATGCACCATGCATCGAGATCTCATCCCCTCCTGC
TAGTGTTGCTACTGCTACTACTGATGGTTCTGAATTAGGCGGAACAATAAGC
AAGAGAAGGCCCATTGCCGTTAGCAGGGCTGAACAACGTTGGTTGATGTC
GACCAAAAGCCGACGACCAGCTGCTGACAACAAGAAGTTATATGCGGATG
TCGACTCCACGATCCAGCACTTGCAACTACAAGCAACCATGAACGGCAAC
TGGATGTGGCCTTATAAACAGGAGGGCAGCACCTCTCACTACATTAGCTTA
CAAGATGATAAGAGGATGCAGACGAAACCATTGTCGTCGCCATCTTTGCCA
GGTTCCATCTGTGAAAGAGCTTTAGGCCTGCACGTGCATGACAGCTTGTCT
ATCGCAAGTATCACAAGCCATTCAAATATGGCACGGAGATGGAATAACCTA
GACTGGTGCAGAGTGGAGAGATGCCCTAACATTGAAGGTGTTGTTTTCACT
CCACCTAGTGATCCTGCTTGTGATAAAATTTTCTGGTACCTGAAGACATTCT
GGGCATCCCAACTAGCAAAGGCGCAGCACATCTGGGGCTGGGGCACAACG
GACCAGCTGCATTTTGAGCCTGATGATAAGTCATTCTACCTGCAAGTGCTAC
ACCTAGATTGCTGCCCCAGGTTGATACATGTGCTTTCCTCATATGACAACAG
TCCTTCCTATGCATACCGTTGGTTGGAAACCCTTGAGATCGTGTGTTGTGGT
GACCTCAAGGATGTCTTCCGAGTGGATGATAATAATCAAGAGCTTCTAAAA
ACTATAGAATTCGAAGAACTGAAGCACATCCACCTGCACGAGCTGCCCTCT
CTGCAACGCATCTGCGGGCATAGGATTGTGGCGCCCAAGCTCGAGACCATC
AAAATCCGAGGCTGCTGGAGCCTCACCCGCCTACCGGCTGTTGGTCTTGAC
AGTACCCGCAAGCCCAAGGTGGATTGTGAGAAGGAATGGTGGGATGGCCT
GGAGTGGGACGGGTTGGAGAACGGACACCATCCTTCCCTCTTTGAGCCCA
CCCACTCACGCTACTACAAGAAGAAACTGCCCAGAGGTTCCATGCTCAGG
TGATCGATATATACGTCCATACACTACATATTTGATACTGTAATCATCTACGTA
TCATTCCCTTTCCCTCATCCAGTGGCTCATCAAGTTCTTCAGCTCTATCTCTA
TTATATATGTCCTTATTTGGGGCTGGTGTTCAAAGTGCTCGCTCATTTCATCA
TATCATTTGCAACGGCATCAATATCTAATGCATGATGAGTAAGTACATCTCCG
GCCGGCATCCAGACGCTCGTCATCTGCTTGTTACTAGCTTGGATTCTGTTCA
TTTCTGCTTCTTTTGTTCTGTGTGAAAATTAATAAATGGATGCTCAGCTGCC
AGCTGGTGTGTGAGCTCTGCATCTATCTGCTAAGTTATTTTCTCCATACATGC
TTGCTTTCCGTTTGGCCGGTTAGTCGATCTGCTTGTTCGACCGATCCATCCA
TCGGATTACTACGTATGCTTCATCTTTCTGGATAGATGATCATACATGTATATG
TGAAAACTTTGATCTCTAGCACCGTTCTGGTGTGGTGTGCTTCCTAGAGGT
CGACGAATAAATCATCAGCTTCAGCGGCATGAGAAGATCCATTGGGTGATC
CTCGTGCAGTCGAGTTGCTGTTGTGCACTTGTTTGTGGGTGCGTGTGTGTT
CGTGTGATTTGTCAGCATATGTGGACTGACCGAGTTTGTACTCCTAATTACT
TCACTTGTATGAACAGAAACAATGATGATGATTGTGAGTGTGACATATATTT
TC
>BPH33.2 protein
(SEQ ID NO. 3)
MPPAEWTMIIADSIDEAAGWVIEFLEDTSKGNVMYFHGWHGLGASAVLRAVA
KRLTMRPSPSQGRRRWEKIIHVDCSLWQSKRALQKAIAQELQLPRSLMALFD
QHDEEDDFSGVDQGMRGVVPLVTQAILSDLVNHTFLVIFHNGSDSYIDLQECG
VPVITGLLNKTVLWTSRSSFRIIFTNFVSEDRHKLAGLSDAAIFADSIGNILGML
LHQEAEEVARYTGVPQSAGMSTELVKKCIMYQLMLRQQHVDYTQHWDTHA
GNYWVCSGIIQTSSDTTSSTSCHSSSPWEIAQALYNNLILEFLTRDNYNMKLIS
VEEIQSTLRLPSDFVDESSFFRTCDAAGNNNVDATTACCRQKSLEAKMFQHPS
ARSLRVIHLFDCTFSFASPPFLSCSSLRFLLLDHCKDKHNLGSAPNSTSAGDTE
KETSISSGACFQKLWVLDLSYTDWYWLLSVEAQDLMVELRELNVKGVKHWS
ISHLLRDDNNSSTGVGSSTKPLGLLNLVKLQVTTEPITEDQHQSQVWKEDQVA
ATLFPNLSSCKIVKTIILDGCFELKRIDPHVLPPSLESFSFSSSSNDNDVHVSAKI
ESISFRGCTQLKSVLLRGLFLRLKQLDVSETCIKTLDLRAMRGNGSLKELFLLG
CKELRAILWPKQDVSLEVLHIDTSSTELDHATGVEESSSFSPVEFKWYISVRDR
RLLRSLNDTKYPSDAPCIEISSPPASVATATTDGSELGGTISKRRPIAVSRAEQRW
LMSTKSRRPAADNKKLYADVDSTIQHLQLQATMNGNWMWPYKQEGSTSHYI
SLQDDKRMQTKPLSSPSLPGSICERALGLHVHDSLSIASITSHSNMARRWNNL
DWCRVERCPNIEGVVFTPPSDPACDKIFWYLKTFWASQLAKAQHIWGWGTTD
QLHFEPDDKSFYLQVLHLDCCPRLIHVLSSYDNSPSYAYRWLETLEIVCCGDL
KDVFRVDDNNQELLKTIEFEELKHIHLHELPSLQRICGHRIVAPKLETIKIRGCW
SLTRLPAVGLDSTRKPKVDCEKEWWDGLEWDGLENGHHPSLFEPTHSRYYKK
KLPRGSMLR
H99:
(SEQ ID NO. 12)
CACTGTGGTTACAACAGAGGTTCAAGGAAATGAAAGAAATGCAAAGCAGC
AGCAGCCGTGATGACAGGCAGAAACGTGAGCAGCAACGAGAAGAGA
H79:
(SEQ ID NO. 13)
CCGTGAGTTCACTTGTAAGTAAGTCAAATAAATAATAAACCCATTTAGATAT
ACAAGTGGGTCGATCCATAAATATATTAATAGGTCAAATTAATAGATAACTCG
CTGGTCAAATCGTAC
33-3:
(SEQ ID NO. 14)
TGTGGCAGAGCAAGAGGGCCCTGCAGAAGGCCATCGCCCAGGAGCTGCA
GCTTCCTCGGTCGCTGATGGCCTTGTTCGACCAGCACGACGAGGAGGATGA
TTTCAGTGGGGTGGATCAGGGCATGAGAGGGGTGGTTCCACTTGTCACAC
AGGCAATCTTGAGCGACCTGGTAAACCACACATTCCTGGTGATCTTCCACA
ATGGGAGCGACAGCTACATTGATCTACAGGAGTGTGGTGTCCCTGTAATAA
CGGGACTCTTGAACAAAACGGTGTTGTGGACCTCCCGAAGCAGCTTTCGG
ATTATCTTCACTAATTTCGTAAGCGAGG
33-4:
(SEQ ID NO.14)
GCTTTCGGATTATCTTCACTAATTTCGTAAGCGAGGATCGGCATAAACTTGC
TGGGCTGAGTGATGCCGCTATATTTGCTGATTCTATTGGAAACATATTGGGA
ATGCTTTTGCATCAAGAGGCAGAGGAGGTTGCCAGGTACACCGGTGTCCCT
CAATCTGCTGGCATGAGCACCGAGCTTGTCAAGAAGTGCATCATGTATCAG
TTGATGCTAAGACAACAACATGTGGACTACACCCAACACTGGGATACCCAC
GCAGGTAACTATTGGGTATGTAGTGGAATCATACAAACCTCTTCAGACACC
ACTAGTAGTACAAGTIGTCATAGCTCATCGCCATGGGAGATAGCCCAAGCT
CTTTATAATAACTTGATCTTGGAGTTTTTGACCAGGGACAATTACAACATGA
AACTAATTTCTGTGGAAGAAATACAGAGTACCCTACGACTGCCTTCTGATTT
TGTGGACGAGTCGTCCTTCTTCCGGACATGTGATGCTGCTGGCAATAACAA
TGTGGACGCAACTACAGCTTGTTGTAGACAGAAATCACTGGAGGCCAAAA
TGTTTCAACATCCATCTGCACGCAGTTTGCGTGTGATACATCTCTTCGATTG
TACCTTCAGTTTTGCATCTCCACCCTTCCTCTCTTGCAGCAGCCTAAGATTC
CTCCTACTTGACCATTGCAAAGACAAACACAACCTCGGCTCAGCACCAAAT
AGTACTAGTGCTGGAGACACTGAAAAGGAGACTAGTATTAGTAGTGGAGCA
TGTTTCCAGAAGCTATGGGTGCTTGACCTGAGTTACACAGATTGGTATTGGC
TGCTATCAGTAGAGGCACAAGATCTGATGGTTGAGCTTAGGGAGCT

In addition to the above embodiments, the present disclosure may also have other implementations. All technical solutions derived from equivalent substitution or equivalent transformation should be considered as falling within the scope of protection defined by the present disclosure.

What is claimed is:

1. A recombinant vector, a recombinant bacterium, a expression cassette or a transgenic cell line, wherein the recombinant vector, the recombinant bacterium, the expression cassette or the transgenic cell line all comprise the rice brown planthopper resistance gene BPH33.2; a nucleotide sequence of the gene BPH33.2 is shown in SEQ ID NO.1.

2. The recombinant vector, the recombinant bacterium, the expression cassette or the transgenic cell line according to claim 1, wherein a cDNA sequence of the gene BPH33.2 is shown in SEQ ID NO.2.

3. The recombinant vector, the recombinant bacterium, the expression cassette or the transgenic cell line according to claim 1, wherein an amino acid sequence of a protein the gene BPH33.2 encodes is shown in SEQ ID NO.3.

4. A method of preparing plant with brown planthopper resistance, wherein the method is a transgenic method or a hybridization method.

5. The method of preparing plant with brown planthopper resistance according to claim 4, wherein the transgenic method comprises:
transferring an expression cassette containing the nucleotide sequence shown in SEQ ID NO.1 or SEQ ID NO.2 into an insect-susceptible plant variety to obtain transgenic rice with brown planthopper resistance.

6. The method of preparing plant with brown planthopper resistance according to claim 5, the transgenic method further comprises:
1) transforming polynucleotides containing the gene BPH33.2 into cells of plant callus to obtain transformed plant cells; the nucleotide sequence of the gene BPH33.2 is as shown in SEQ ID NO. 1 or SEQ ID NO. 2;
2) regenerating the transformed plant cells into plant to obtain regenerated plant;
3) culturing the regenerated plant and allowing expression of the polynucleotides, and harvesting T1 generation seeds;
4) sowing the T1 generation seeds, detecting the gene BPH33.2 using marker primers 33-3 and 33-4, and harvesting genetically pure T2 generation seeds;
the marker primers 33-3 are as follows:
left end primer:
5′-CCTCGCTTACGAAATTAGTG-3′,
right end primer:
5′-TGTGGCAGAGCAAGAGGG-3′;
the marker primers 33-4 are as follows:
left end primer:
5′-GCTTTCGGGATTATCTTCACTA-3′,
right end primer:
5′-AGCTCCCTAAGCTCAACCA-3′;
5) sowing the T2 generation seeds to obtain transgenic plant with brown planthopper resistance.

7. The method of preparing plant with brown planthopper resistance according to claim 4, wherein the hybridization method comprises:
crossing a plant having the brown planthopper resistance gene BPH33.2 with other plant to generate a progeny plant with brown planthopper resistance.

8. The method of preparing plant with brown planthopper resistance according to claim 4, wherein the plant is rice.

9. An application of molecular markers H99, H79, 33-3 and 33-4 in the breeding of brown planthopper resistant rice, wherein nucleotide sequences of the molecular markers H99, H79, 33-3, 33-4 are respectively shown in SEQ ID NO.12-15.

10. The application of molecular markers H99, H79, 33-3 and 33-4 in the breeding of brown planthopper resistant rice according to claim 9, comprising:
amplifying genomic DNAs of rices to be examined by marker primers, and detecting amplification products; the marker primers are H99 marker primers, H79 marker primers, 33-3 marker primers and 33-4 marker primers;
the H99 marker primers are as follows:
left end primer:
5′-CACTGTGGGTTACAACAGAGGT-3′,
right end primer:
5′-TCTCTTCTCGTTGCTGCTCA-3′;
the H79 marker primers are as follows:
left end primer:
5′-CCGTGAGTTCACTTGTAA-3′,
right end primer:
5′-GTACGATTTGACCAGCGAG-3′
the marker primers 33-3 are as follows:
left end primer:
5′-CCTCGCTTACGAAATTAGTG-3′,
right end primer:
5′-TGTGGCAGAGCAAGAGGG-3′;
the marker primers 33-4 are as follows:
left end primer:
5′-GCTTTCGGGATTATCTTCACTA-3′,
right end primer:
5′-AGCTCCCTAAGCTCAACCA-3′;
if a DNA fragment of 96 bp can be amplified by H99 marker primers, or a DNA fragment of 120 bp can be amplified by the H79 marker primers, or a DNA fragment of 330 bp can be amplified by the 33-3 marker primers, or a DNA fragment of 868 bp can be amplified by the 33-4 marker primers, all of them indicate a presence of a brown planthopper resistance locus BPH33.2 in rice insect-resistant varieties; if only a 112 bp DNA fragment can be amplified by the H99 marker primers, or only a 129 bp DNA fragment can be amplified by the H79 marker primers, or no DNA fragment can be amplified by the 33-3 marker primers, or no DNA fragment can be amplified by the 33-4 marker primers, all of them indicate an absence of a brown planthopper resistance locus BPH33.2 in rice insect-resistant varieties.


SEQUENCE LISTING (15 sequences)

SEQ ID NO: 1 - DNA, 8674 bp, Oryza sativa
  source 1..8674: mol_type = genomic DNA
gaattcaccttccgcgcacgcaaaatggagtgatgtattttcttacgattaattaggtatttactatttttttgaaaaatagattaatatgattctttaaagcaactttcgtatagaaactttttgcaaaatacatattgtttagcggtttgaaaagcgtacacgcggaacacgacggaggtgagttgggaaaaatgggggaagaacacaacctaagtcttaaactctcctccacggctcctctatcgagaaccactcattctctccctctcctatcttctctctctcactagatccacacacgagctctccttatccttcacttgccaccgtttctcctcgtgtagaagcaaccacacggcttgccttggcttggcgacggcgacttcgaacacatggcattggatgagcagtgaaggggaaggggtgggtcggcattatcgaacgagggccagagcttgggactagcgacaacaatggcgactgcggcaagggcagggtggtggtggcaaacaatgggctgagctcaggatctgtggtgaacgaacatctatgtgggttgtttacaacaatgttgtggccctagatctgttggagtgaaggctagatctaccatcgaggaggtcagatctggatggtggctattacctctctcgatgccgccgctagtgggcagatccaagccatcatgcttcatgtctcctgcggcgggtggcggtagatcggggtactgggatggtggttcactactagagaaatgcacttcggtgcctgttgggaagaccccttagatgtcggttttcctaaccagcaccatgaaggccgcactgattaccggcaccaatgtcattttaaaaaccgacaccaatatggaatcaacgagcaaaagaaaaaaaagccagccggctctagccgattaactcccacatgaacccacatgaacaacaaacaaatgatgaacagattccacatcaacagatgaacaagagcgacacaaaatctcacaccaattatcatagcaacaaatgaactcttgcatgaacaagaacgtatctcaaacaaatcacaaagtgggagggagggggagagatggatccactggccgccgatgccgtcacacctccagatccaacacgggccacctccgtcccgccaccgcacgccgcctcccgccgccaaggggcgcagaccgcctccgtcccgtcgccgcacgctgccgcacttcgcctaccaccgccgaggggcgcggaccacctccgtcctgccgccgcatgccgcctccgatcgccgaggggcacgggctgcctccttcccgccgccgcacaccaccgatctcgccgccgaggggcatgcaagacgacggtgagaaggctagatccagctgctgggaccctgtgaactccggtggtggtggaacctccctggcttcttagggacagcgacaacaacttcaacgtcgaccacatgattttcgcatgccttatacttgtaagcggggcggctgggcgcccatgaaaaatcatttttttctacctgcgaatacccatcttgtagtggtgctagattagcaaacctgttttctccttcttttttttggggggggggggtgtttgaactatggtggcttaggtagcctacatgccaatcaaagtttcaaatttagtttaactccaagggagaatgtattctatcgaatttagaaaaaatgtcaaaattcatgatttatttaattgtttgtggaattatgtttgaaattttgtaatttatgttaccccgaaatgtttatgcattatgagatagttaaccctatactccctccggttctatattaattgacgttttgaataaggttgaggctaaacttttataactttgaccatcaataactttaaaaatatttaatttaaagaaaatagaataacatatataaatttttctttcaaagcactataataaaagtaaacatgcatttatttattgtatatattataatagaaaaataagatcaaagatatatcttgtagactgtgtcattgtccaaaacgtcaattaaaatgatactggagggagggagtatagggtatcatattctaaccttggggttaacaagacacagttgcttttgtatcctggattgacgacgtacatcccttttgctttatcttttgctttttgcactttaatcaaggaggcgaagcaaagaatacgacgattagccaactactaactccaaattatctatagccaatttaatagttgatttatacaacaattgcttacaatactattaatatgtgatcccacctgtcatagaagcatatgttatgtcttgaagtccgtgttacagctgtctacagatctgtagcccactgcttttctctctacttttttatctctttaaaatatatatgtttatagctggcttatagtctgctattgtacctcctctcagatgatgcattcaggtacagcttaggacgtacatatagcatatcgatttgcggctttgcagcaaatgcaaagcaaagaaaaatactaaagcaaacatatcgatcgtacgcaaggaataatagatcgacgaagcatgttgtaataatgcaagcattctagacgacagtacgtttttacaaggcttctgctagctctagcccagaacatcgatctatcgatcatattgcatccagtttgatcgtttcaatttatgctcgccctcgcaccaacaatggcttagctaatccccatgtactcgatctctgtcggcaaacatcgtgattaaggtaaactagctacatcatatcgtgcagtttaattgcgctctctctctctctctctccccctatgtgtgtgtgttgatgcatcaacatgcatatatatatatatatatatatatatatatatatatatatatatatatatatatatatatatatatatatatatatatatatatatatatatatatatatatatatatatatatatatatatatgtatgtatgtacaaaatcatctcagttttataagctttaattttagttagatctctagctatgtggtatattatttaatttggttttctacgggatgttcacacatatataaatatatgaaatgcttgcatgctgttgtaattctggcttaatttgtacgtttcgtttatatgaaaagatgatttagtttatataacatgcatgtaaacacttctccattatcctcaaaacaatccctagggagaacgaattaaagatatatatgcatccgtgtagtatatcattttcgtcgttgattattaatcaaattaaaatacacttgcattcacttgtgtccatttgaaacccaggcgcatgttgggccgggccactagtacaaaattaatcatgtacagtaactaatttattaagtatatatgttttacatgcgtatatacatgaactactgtatctataatgtgcaggctggagcgagtaattaaataatcaggagtgggctggagaaatcgatccatcaggcatctatatatcatgggttaattaattacagggcttgcttcaacatatatatgtattctctgctgtcgatccatttctaattaaggagcgtttaatcaactgaaagaagatcgattgagattatatgatcgatgaagctgcccgcttaaccgcaattcaacattaattgaagagagccaggctacgcagatcaactcaacatgccaccagcggaggtaattaaatcagatcggtgatattgtttttatttttgaatgaaagttatttcacgaccacgcacgtgaattaaacaaggaaaactttactgctattttagatttccaatttggacgcaattttttgaaacacgctttcattgtcatatttggctttagaggtgtttttttttctaacatctcatttgcaaacaatcctctcggagcaaacttcttctatttgtggaagaaataaattgcaaaaaggataattttaccatccttagaagataacatgaggtaccatattttatactataaattttggcacatcaagtattatgtatctcaatgtataaaagaaataattttagtgtaaacttttgataataacaagtaattggtcaagaaccgtaaaatttctcacactccacctgttcaaaattcaaatgctatgtttggctagttgcagagagaaaaaatatatatagtgtaattaataactttattcgtaacattatatatatgccggtttgcactctacctttttgtggtctattacactttattctattgtaattaataaatacaccaccgagaaaactgggcgtttctttagaattttatttgtgataaaagattttagtgtgtttgtttagctatgaacttgtttcattgtgacagagggaataaatatactaatgaaaataattaaaactagctaattcagctttgcctgtattgatcgcggaatcaccgaccgagaagctctctagttctagggcaggtttgaattaattagtggtattaatgaatgattaattattgcagtggaccatgatcatagcagacagcatcgatgaagcagcaggctgggtaattgaatttctggaggatacaagcaaggggaatgtgatgtatttccatggctggcatggactgggggcttcagccgtcctcagagcagtcgccaaaagactgacgatgaggccatcgccatcgcaaggaagaagaaggtgggagaagatcatccacgttgattgctctctgtggcagagcaagagggccctgcagaaggccatcgcccaggagctgcagcttcctcggtcgctgatggccttgttcgaccagcacgacgaggaggatgatttcagtggggtggatcagggcatgagaggggtggttccacttgtcacacaggcaatcttgagcgacctggtaaaccacacattcctggtgatcttccacaatgggagcgacagctacattgatctacaggagtgtggtgtccctgtaataacgggactcttgaacaaaacggtgttgtggacctcccgaagcagctttcggattatcttcactaatttcgtaagcgaggatcggcataaacttgctgggctgagtgatgccgctatatttgctgattctattggaaacatattgggaatgcttttgcatcaagaggcagaggaggttgccaggtacaccggtgtccctcaatctgctggcatgagcaccgagcttgtcaagaagtgcatcatgtatcagttgatgctaagacaacaacatgtggactacacccaacactgggatacccacgcaggtaactattgggtatgtagtggaatcatacaaacctcttcagacaccactagtagtacaagttgtcatagctcatcgccatgggagatagcccaagctctttataataacttgatcttggagtttttgaccagggacaattacaacatgaaactaatttctgtggaagaaatacagagtaccctacgactgccttctgattttgtggacgagtcgtccttcttccggacatgtgatgctgctggcaataacaatgtggacgcaactacagcttgttgtagacagaaatcactggaggccaaaatgtttcaacatccatctgcacgcagtttgcgtgtgatacatctcttcgattgtaccttcagttttgcatctccacccttcctctcttgcagcagcctaagattcctcctacttgaccattgcaaagacaaacacaacctcggctcagcaccaaatagtactagtgctggagacactgaaaaggagactagtattagtagtggagcatgtttccagaagctatgggtgcttgacctgagttacacagattggtattggctgctatcagtagaggcacaagatctgatggttgagcttagggagctaaatgtgaagggggtcaagcattggagcataagccatctactccgtgatgacaacaattctagtactggtgtcggaagcagcaccaagccccttgggctactcaaccttgtcaagctccaagtcacaaccgaaccaataactgaagatcaacatcagtcacaagtatggaaggaagatcaggtagcagcaacattattcccgaacctatctagctgcaaaatcgtaaagacgatcattcttgatggttgttttgagttgaagcgaattgaccctcacgtcctgccaccatcactcgagtcatttagcttctccagcagcagcaatgacaatgatgttcatgtctcggccaagatagagagcatctccttccggggatgcacccagttgaagagtgtgcttttgagaggactatttctaaggctcaagcagctagacgtgtcagaaacttgtattaaaaccctcgacctacgtgcaatgcgaggcaacgggtctctcaaggagctatttctgcttggatgcaaggagcttcgtgcaatactatggccaaaacaagatgtatcattggaggttctgcacatcgacactagtagcacagaacttgatcatgctacaggtgtagaagaatcatcatcattctcacctgttgaatttaaatggtatatttcagtgagggacagaaggctccttcgctcgctaaatgatacaaaatatccctcggatgcaccatgcatcgagatctcatcccctcctgctagtgttgctactgctactactgatggttctgaattaggcggaacaataagcaagagaaggcccattgccgttagcagggctgaacaacgttggttgatgtcgaccaaaagccgacgaccagctgctgacaacaagaagttatatgcggatgtcgactccacgatccagcacttgcaactacaagcaaccatgaacggcaactggatgtggccttataaacaggagggcagcacctctcactacattagcttacaagatgataagaggatgcagacgaaaccattgtcgtcgccatctttgccaggttccatctgtgaaagagctttaggcctgcacgtgcatgacagcttgtctatcgcaagtatcacaagccattcaaatatggcacggagatggaataacctagactggtgcagagtggagagatgccctaacattgaaggtgttgttttcactccacctagtgatcctgcttgtgataaaattttctggtacctgaagacattctgggcatcccaactagcaaaggcgcagcacatctggggctggggcacaacggaccagctgcattttgagcctgatgataagtcattctacctgcaagtgctacacctagattgctgccccaggttgatacatgtgctttcctcatatgacaacagtccttcctatgcataccgttggttggaaacccttgagatcgtgtgttgtggtgacctcaaggatgtcttccgagtggatgataataatcaagagcttctaaaaactatagaattcgaagaactgaagcacatccacctgcacgagctgccctctctgcaacgcatctgcgggcataggattgtggcgcccaagctcgagaccatcaaaatccgaggctgctggagcctcacccgcctaccggctgttggtcttgacagtacccgcaagcccaaggtggattgtgagaaggaatggtgggatggcctggagtgggacgggttggagaacggacaccatccttccctctttgagcccacccactcacgctactacaagaagaaactgcccagaggttccatgctcaggtatgtacatatgtctgtgtgcatacatgattgttacttgggtatcattccttgcgcgcacatacatacatacgtagaaacttatatagctcagtactttagttactccatcatatatatatatatatatatatatgatcgagcttttccctttttttttaattttgtctgacttggcgtgctttcttttcttcttaaattcaggtgatcgatatatacgtccatacactacatatttgatactgtaatcatctacgtatcattccctttccctcatccagtggctcatcaagttcttcagctctatctctattatatatgtccttatttggggctggtgttcaaagtgctcgctcatttcatcatatcatttgcaacggcatcaatatctaatgcatgatgagtaagtacatctccggccggcatccagacgctcgtcatctgcttgttactagcttggattctgttcatttctgcttcttttgttctgtgtgaaaattaataaatggatgctcagctgccagctggtgtgtgagctctgcatctatctgctaagttattttctccatacatgcttgctttccgtttggccggttagtcgatctgcttgttcgaccgatccatccatcggattactacgtatgcttcatctttctggatagatgatcatacatgtatatgtgaaaactttgatctctagcaccgttctggtgtggtgtgcttcctagaggtcgacgaataaatcatcagcttcagcggcatgagaagatccattgggtgatcctcgtgcagtcgagttgctgttgtgcacttgtttgtgggtgcgtgtgtgttcgtgtgatttgtcagcatatgtggactgaccgagtttgtactcctaattacttcacttgtatgaacagaaacaatgatgatgattgtgagtgtgacatatattttc

SEQ ID NO: 2 - DNA, 4470 bp, Oryza sativa
  source 1..4470: mol_type = other DNA
aaatgcaaagcaaagaaaaatactaaagcaaacatatcgatcgtacgcaaggaataatagatcgacgaagcatgttgtaataatgcaagcattctagacgacagtacgtttttacaaggcttctgctagctctagcccagaacatcgatctatcgatcatattgcatccagtttgatcgtttcaatttatgctcgccctcgcaccaacaatggcttagctaatccccatgtactcgatctctgtcggcaaacatcgtgattaaggctggagcgagtaattaaataatcaggagtgggctggagaaatcgatccatcaggcatctatatatcatgggttaattaattacagggcttgcttcaacatatatatgtattctctgctgtcgatccatttctaattaaggagcgtttaatcaactgaaagaagatcgattgagattatatgatcgatgaagctgcccgcttaaccgcaattcaacattaattgaagagagccaggctacgcagatcaactcaacatgccaccagcggagtggaccatgatcatagcagacagcatcgatgaagcagcaggctgggtaattgaatttctggaggatacaagcaaggggaatgtgatgtatttccatggctggcatggactgggggcttcagccgtcctcagagcagtcgccaaaagactgacgatgaggccatcgccatcgcaaggaagaagaaggtgggagaagatcatccacgttgattgctctctgtggcagagcaagagggccctgcagaaggccatcgcccaggagctgcagcttcctcggtcgctgatggccttgttcgaccagcacgacgaggaggatgatttcagtggggtggatcagggcatgagaggggtggttccacttgtcacacaggcaatcttgagcgacctggtaaaccacacattcctggtgatcttccacaatgggagcgacagctacattgatctacaggagtgtggtgtccctgtaataacgggactcttgaacaaaacggtgttgtggacctcccgaagcagctttcggattatcttcactaatttcgtaagcgaggatcggcataaacttgctgggctgagtgatgccgctatatttgctgattctattggaaacatattgggaatgcttttgcatcaagaggcagaggaggttgccaggtacaccggtgtccctcaatctgctggcatgagcaccgagcttgtcaagaagtgcatcatgtatcagttgatgctaagacaacaacatgtggactacacccaacactgggatacccacgcaggtaactattgggtatgtagtggaatcatacaaacctcttcagacaccactagtagtacaagttgtcatagctcatcgccatgggagatagcccaagctctttataataacttgatcttggagtttttgaccagggacaattacaacatgaaactaatttctgtggaagaaatacagagtaccctacgactgccttctgattttgtggacgagtcgtccttcttccggacatgtgatgctgctggcaataacaatgtggacgcaactacagcttgttgtagacagaaatcactggaggccaaaatgtttcaacatccatctgcacgcagtttgcgtgtgatacatctcttcgattgtaccttcagttttgcatctccacccttcctctcttgcagcagcctaagattcctcctacttgaccattgcaaagacaaacacaacctcggctcagcaccaaatagtactagtgctggagacactgaaaaggagactagtattagtagtggagcatgtttccagaagctatgggtgcttgacctgagttacacagattggtattggctgctatcagtagaggcacaagatctgatggttgagcttagggagctaaatgtgaagggggtcaagcattggagcataagccatctactccgtgatgacaacaattctagtactggtgtcggaagcagcaccaagccccttgggctactcaaccttgtcaagctccaagtcacaaccgaaccaataactgaagatcaacatcagtcacaagtatggaaggaagatcaggtagcagcaacattattcccgaacctatctagctgcaaaatcgtaaagacgatcattcttgatggttgttttgagttgaagcgaattgaccctcacgtcctgccaccatcactcgagtcatttagcttctccagcagcagcaatgacaatgatgttcatgtctcggccaagatagagagcatctccttccggggatgcacccagttgaagagtgtgcttttgagaggactatttctaaggctcaagcagctagacgtgtcagaaacttgtattaaaaccctcgacctacgtgcaatgcgaggcaacgggtctctcaaggagctatttctgcttggatgcaaggagcttcgtgcaatactatggccaaaacaagatgtatcattggaggttctgcacatcgacactagtagcacagaacttgatcatgctacaggtgtagaagaatcatcatcattctcacctgttgaatttaaatggtatatttcagtgagggacagaaggctccttcgctcgctaaatgatacaaaatatccctcggatgcaccatgcatcgagatctcatcccctcctgctagtgttgctactgctactactgatggttctgaattaggcggaacaataagcaagagaaggcccattgccgttagcagggctgaacaacgttggttgatgtcgaccaaaagccgacgaccagctgctgacaacaagaagttatatgcggatgtcgactccacgatccagcacttgcaactacaagcaaccatgaacggcaactggatgtggccttataaacaggagggcagcacctctcactacattagcttacaagatgataagaggatgcagacgaaaccattgtcgtcgccatctttgccaggttccatctgtgaaagagctttaggcctgcacgtgcatgacagcttgtctatcgcaagtatcacaagccattcaaatatggcacggagatggaataacctagactggtgcagagtggagagatgccctaacattgaaggtgttgttttcactccacctagtgatcctgcttgtgataaaattttctggtacctgaagacattctgggcatcccaactagcaaaggcgcagcacatctggggctggggcacaacggaccagctgcattttgagcctgatgataagtcattctacctgcaagtgctacacctagattgctgccccaggttgatacatgtgctttcctcatatgacaacagtccttcctatgcataccgttggttggaaacccttgagatcgtgtgttgtggtgacctcaaggatgtcttccgagtggatgataataatcaagagcttctaaaaactatagaattcgaagaactgaagcacatccacctgcacgagctgccctctctgcaacgcatctgcgggcataggattgtggcgcccaagctcgagaccatcaaaatccgaggctgctggagcctcacccgcctaccggctgttggtcttgacagtacccgcaagcccaaggtggattgtgagaaggaatggtgggatggcctggagtgggacgggttggagaacggacaccatccttccctctttgagcccacccactcacgctactacaagaagaaactgcccagaggttccatgctcaggtgatcgatatatacgtccatacactacatatttgatactgtaatcatctacgtatcattccctttccctcatccagtggctcatcaagttcttcagctctatctctattatatatgtccttatttggggctggtgttcaaagtgctcgctcatttcatcatatcatttgcaacggcatcaatatctaatgcatgatgagtaagtacatctccggccggcatccagacgctcgtcatctgcttgttactagcttggattctgttcatttctgcttcttttgttctgtgtgaaaattaataaatggatgctcagctgccagctggtgtgtgagctctgcatctatctgctaagttattttctccatacatgcttgctttccgtttggccggttagtcgatctgcttgttcgaccgatccatccatcggattactacgtatgcttcatctttctggatagatgatcatacatgtatatgtgaaaactttgatctctagcaccgttctggtgtggtgtgcttcctagaggtcgacgaataaatcatcagcttcagcggcatgagaagatccattgggtgatcctcgtgcagtcgagttgctgttgtgcacttgtttgtgggtgcgtgtgtgttcgtgtgatttgtcagcatatgtggactgaccgagtttgtactcctaattacttcacttgtatgaacagaaacaatgatgatgattgtgagtgtgacatatattttc

SEQ ID NO: 3 - protein, 1072 aa, Oryza sativa
MPPAEWTMIIADSIDEAAGWVIEFLEDTSKGNVMYFHGWHGLGASAVLRAVAKRLTMRPSPSQGRRRWEKIIHVDCSLWQSKRALQKAIAQELQLPRSLMALFDQHDEEDDFSGVDQGMRGVVPLVTQAILSDLVNHTFLVIFHNGSDSYIDLQECGVPVITGLLNKTVLWTSRSSFRIIFTNFVSEDRHKLAGLSDAAIFADSIGNILGMLLHQEAEEVARYTGVPQSAGMSTELVKKCIMYQLMLRQQHVDYTQHWDTHAGNYWVCSGIIQTSSDTTSSTSCHSSSPWEIAQALYNNLILEFLTRDNYNMKLISVEEIQSTLRLPSDFVDESSFFRTCDAAGNNNVDATTACCRQKSLEAKMFQHPSARSLRVIHLFDCTFSFASPPFLSCSSLRFLLLDHCKDKHNLGSAPNSTSAGDTEKETSISSGACFQKLWVLDLSYTDWYWLLSVEAQDLMVELRELNVKGVKHWSISHLLRDDNNSSTGVGSSTKPLGLLNLVKLQVTTEPITEDQHQSQVWKEDQVAATLFPNLSSCKIVKTIILDGCFELKRIDPHVLPPSLESFSFSSSSNDNDVHVSAKIESISFRGCTQLKSVLLRGLFLRLKQLDVSETCIKTLDLRAMRGNGSLKELFLLGCKELRAILWPKQDVSLEVLHIDTSSTELDHATGVEESSSFSPVEFKWYISVRDRRLLRSLNDTKYPSDAPCIEISSPPASVATATTDGSELGGTISKRRPIAVSRAEQRWLMSTKSRRPAADNKKLYADVDSTIQHLQLQATMNGNWMWPYKQEGSTSHYISLQDDKRMQTKPLSSPSLPGSICERALGLHVHDSLSIASITSHSNMARRWNNLDWCRVERCPNIEGVVFTPPSDPACDKIFWYLKTFWASQLAKAQHIWGWGTTDQLHFEPDDKSFYLQVLHLDCCPRLIHVLSSYDNSPSYAYRWLETLEIVCCGDLKDVFRVDDNNQELLKTIEFEELKHIHLHELPSLQRICGHRIVAPKLETIKIRGCWSLTRLPAVGLDSTRKPKVDCEKEWWDGLEWDGLENGHHPSLFEPTHSRYYKKKLPRGSMLR

SEQ ID NO: 4 - DNA, 21 bp, synthetic construct
  source 1..21: mol_type = other DNA
cactgtggttacaacagaggt

SEQ ID NO: 5 - DNA, 20 bp, synthetic construct
  source 1..20: mol_type = other DNA
tctcttctcgttgctgctca

SEQ ID NO: 6 - DNA, 18 bp, synthetic construct
  source 1..18: mol_type = other DNA
ccgtgagttcacttgtaa

SEQ ID NO: 7 - DNA, 19 bp, synthetic construct
  source 1..19: mol_type = other DNA
gtacgatttgaccagcgag

SEQ ID NO: 8 - DNA, 20 bp, synthetic construct
  source 1..20: mol_type = other DNA
cctcgcttacgaaattagtg

SEQ ID NO: 9 - DNA, 18 bp, synthetic construct
  source 1..18: mol_type = other DNA
tgtggcagagcaagaggg

SEQ ID NO: 10 - DNA, 21 bp, synthetic construct
  source 1..21: mol_type = other DNA
gctttcggattatcttcacta

SEQ ID NO: 11 - DNA, 19 bp, synthetic construct
  source 1..19: mol_type = other DNA
agctccctaagctcaacca

SEQ ID NO: 12 - DNA, 96 bp, Oryza sativa
  source 1..96: mol_type = genomic DNA
cactgtggttacaacagaggttcaaggaaatgaaagaaatgcaaagcagcagcagccgtgatgacaggcagaaacgtgagcagcaacgagaagaga

SEQ ID NO: 13 - DNA, 120 bp, Oryza sativa
  source 1..120: mol_type = genomic DNA
ccgtgagttcacttgtaagtaagtcaaataaataataaacccatttagatatacaagtgggtcgatccataaatatattaataggtcaaattaatagataactcgctggtcaaatcgtac

SEQ ID NO: 14 - DNA, 330 bp, Oryza sativa
  source 1..330: mol_type = genomic DNA
tgtggcagagcaagagggccctgcagaaggccatcgcccaggagctgcagcttcctcggtcgctgatggccttgttcgaccagcacgacgaggaggatgatttcagtggggtggatcagggcatgagaggggtggttccacttgtcacacaggcaatcttgagcgacctggtaaaccacacattcctggtgatcttccacaatgggagcgacagctacattgatctacaggagtgtggtgtccctgtaataacgggactcttgaacaaaacggtgttgtggacctcccgaagcagctttcggattatcttcactaatttcgtaagcgagg

SEQ ID NO: 15 - DNA, 868 bp, Oryza sativa
  source 1..868: mol_type = genomic DNA
gctttcggattatcttcactaatttcgtaagcgaggatcggcataaacttgctgggctgagtgatgccgctatatttgctgattctattggaaacatattgggaatgcttttgcatcaagaggcagaggaggttgccaggtacaccggtgtccctcaatctgctggcatgagcaccgagcttgtcaagaagtgcatcatgtatcagttgatgctaagacaacaacatgtggactacacccaacactgggatacccacgcaggtaactattgggtatgtagtggaatcatacaaacctcttcagacaccactagtagtacaagttgtcatagctcatcgccatgggagatagcccaagctctttataataacttgatcttggagtttttgaccagggacaattacaacatgaaactaatttctgtggaagaaatacagagtaccctacgactgccttctgattttgtggacgagtcgtccttcttccggacatgtgatgctgctggcaataacaatgtggacgcaactacagcttgttgtagacagaaatcactggaggccaaaatgtttcaacatccatctgcacgcagtttgcgtgtgatacatctcttcgattgtaccttcagttttgcatctccacccttcctctcttgcagcagcctaagattcctcctacttgaccattgcaaagacaaacacaacctcggctcagcaccaaatagtactagtgctggagacactgaaaaggagactagtattagtagtggagcatgtttccagaagctatgggtgcttgacctgagttacacagattggtattggctgctatcagtagaggcacaagatctgatggttgagcttagggagct